(19) United States
(12) Patent Application Publication
(10) Pub. No.: US 2019/0062767 A1
(43) Pub. Date: Feb. 28, 2019

(54) METHOD AND SYSTEM FOR EXPRESSING FOREIGN GENE IN A PLANT

(71) Applicant: NATIONAL INSTITUTE OF ADVANCED INDUSTRIAL SCIENCE AND TECHNOLOGY, Tokyo (JP)

(72) Inventors: Noriho Fukuzawa, Hokkaido (JP); Takeshi Matsumura, Hokkaido (JP)

(21) Appl. No.: 15/691,507

(22) Filed: Aug. 30, 2017

(51) Int. Cl. C12N 15/82

(52) U.S. Cl. CPC C12N 15/8217; C12N 2800/10; C12N 15/8205; C12N 15/8203

(57) ABSTRACT

A method is provided for expressing a foreign gene, comprising the steps of:
(a) producing a plant which functionally expresses an RNA1 genome and/or an RNA2 genome of Cucumber Mosaic Virus (CMV);
(b) introducing a nucleic acid vector into the plant, said nucleic acid vector comprising, arranged in this order:
   (i) a right border sequence (RB) derived from the Agrobacterium T-DNA sequence;
   (ii) an expression promoter sequence workable in the plant;
   (iii) a sequence corresponding to an RNA3 genome of CMV in which a 3b gene, which codes for a 3b protein (coat protein), of CMV has been prevented from being expressed and the foreign gene has been inserted downstream of, and operably linked with, a subgenomic promoter which otherwise promotes the expression of the 3b gene; and
   (iv) a left border sequence (LB) derived from the Agrobacterium T-DNA sequence; and
(c) culturing the plant containing the nucleic acid vector such that the RNA1 and/or RNA2 genome(s) of CMV is functionally expressed by the plant while the foreign gene is expressed transiently from the nucleic acid vector along with the CMV RNA3 genome lacking a region corresponding to the 3b gene.

### TECHNICAL FIELD

The present invention relates to a method and a system for expressing a foreign gene in a plant.

### BACKGROUND ART

Transient expression technique, which can express a target gene in a plant in about a few days to two weeks, is an effective means for producing a substance using a plant.

Conventional transient expression methods using plants are generally categorized into (1) agroinfiltration method, (2) plant virus vector method, (3) magnICON® system, and (4) particle gun method.
(1) The agroinfiltration method involves infecting a plant with Agrobacterium transformed with a T-DNA vector into which a target gene has been inserted, e.g., by introducing a culture of the transformed Agrobacterium into tissues of the plant via physical means (such as syringe injection or vacuum infiltration), such that the target gene is transiently expressed in the plant (Non-Patent Document 1: Grimsley N., Hohn B., Hohn T., and Walden R., “Agroinfection,” an alternative route for viral infection of plants by using the Ti Plasmid., Proc. Natl. Acad. Sci. USA, (1986), 83(10):3282-6). Since Agrobacterium with strong infectability is transferred throughout a plant via physical means (such as injection or infiltration) and caused to infect the plant, the target gene can be expressed evenly in all plant tissues. However, the expression level of the target gene at each cell is limited by the infectability of Agrobacterium and the capabilities of the expression control sequences (e.g., the promoter and the terminator) in the introduced gene. Therefore, it is difficult to improve the total expression level in a plant.
(2) The plant virus vector method involves in-vitro transcribing cDNA of the genome of a plant virus into which a target gene has been inserted, and inoculating the resultant RNA as a vector into a plant to cause infection, such that the target gene is expressed in the plant based on the amplification ability and the systemic transition ability of the virus. Examples of such plant virus vectors include: cucumber mosaic virus (CMV) vector, developed by the present inventors (Patent Document 1: US2007/0143877A); tobacco mosaic virus (TMV) vector (Non-Patent Document 2: Takamatsu N, Ishikawa M, Meshi T, and Okada Y., Expression of bacterial chloramphenicol acetyltransferase gene in tobacco plants mediated by TMV-RNA, EMBO J., (1987), 6(2):307-11); and potato X virus (PVX) vector (Non-Patent Document 3: Baulcombe, D. C., Chapman, S., and Santa Cruz, S., Jellyfish green fluorescent protein as a reporter for virus infectious, Plant J., (1995), 7:1045-1053). Since this method employs the autonomous replication ability of a virus for causing expression of a target gene, it is possible to increase the expression level of the target gene per cell in plant by using a vector having strong amplification ability, such as a CMV- or TMV-based vector. However, it is very difficult to cause infection of the virus vector and expression of the target gene in all cells throughout the plant, since transition of a vector into each tissue cell of the plant depends on the transition ability of the virus. As a result, the plant resulting from introduction of the vector is in a chimeric (mosaic) state composed of a first population of cells, which are infected with the virus and express the target gene, and a second population of cells, which are not infected with the virus and does not express the target gene. Therefore, it is also difficult to improve the total expression level in a plant.
(3) The MagnICON® system is prepared by incorporating, into T-DNA vector, cDNA of the TMV or PVX genome in which a target gene has been inserted. Agrobacterium is transformed with the resultant vector, and a culture of the transformed Agrobacterium is introduced into tissues of a plant via physical means (such as syringe injection or vacuum infiltration), such that the target gene is transiently expressed in the plant (Patent Document 2: US2007/0044170A; Patent Document 3: US2009/0111145A; Patent Document 4: US2007/0044170A). According to this system, it is possible to systemically introduce the vector throughout the plant via physical means (such as injection or infiltration) to cause infection, thereby allowing for expression of the target gene in all tissues of the plant. In addition, since this system employs the autonomous replication ability of a virus for causing expression of the target gene, it is possible to increase the expression level of the target gene per cell in plant. Thus, this system combines advantages of both (1) the agroinfiltration method and (2) the plant virus vector method. However, since this system can only be established on a limited type of plant viruses whose genome is composed of one single-stranded RNA having high amplification ability, such as TMV or PVX, the host plant to be infected by this vector system is quite limited. In addition, since all the genes necessary for replication of the virus (TMV or PVX) must be arranged within the one single-stranded RNA in addition to the target gene to be expressed, the size of the target gene to be inserted is also quite limited.
(4) The particle gun (particle bombardment) method involves injecting metal microparticles coated with nucleic acids to a cell at a high speed to thereby introduce a target gene into a cell (Non-Patent Document 4: Christou P, Particle bombardment, Methods Cell Biol., (1995), 50:375-82). Although it can easily be carried out using various organisms or tissues, this method is not practical due to various problems such as the relatively high cost involved in this method, the possibility that the target gene may be disrupted on introduction, and the extremely low introduction rate into cells in plant.

The present inventors developed a technique involving: preparing a nucleic acid molecule containing a sequence corresponding to the RNA2 genome of cucumber mosaic virus (CMV) in which a gene coding for a 2b protein has partially or entirely been replaced with a foreign gene, and a T-DNA sequence of Agrobacterium functionally linked to the sequence corresponding to the CMV RNA2 genome; introducing the nucleic acid molecule into a host plant which functionally expresses the RNA1 and RNA3 genomes of CMV and the 2b protein of CMV; and culturing the plant such that it expresses the foreign gene. This technique allows for systemic transition of the foreign gene into cells throughout the plant, with improving the expression efficiency of the foreign gene in each cell, thereby achieving increased expression of the foreign gene in the plant as a whole. This technique also expands the freedom of choice for the type of host plant to be used and the size of target gene to be inserted, compared with the MagnICON° system based on TMV or PVX. The present inventors already filed a patent application directed to this technique (Patent Document 5: US2016/0002654A).

### CITATION LIST

### Patent Documents

[Patent Document 1] US2007/0143877A

[Patent Document 2] US2007/0044170A

[Patent Document 3] US2009/0111145A

[Patent Document 4] US2007/0044170A

[Patent Document 5] US2016/0002654A

### Non-Patent Documents

[Non-Patent Document 1] Grimsley et al., PNAS USA, (1986), 83(10):3282-6

[Non-Patent Document 2] Takamatsu et al., EMBO J., (1987), 6(2):307-11

[Non-Patent Document 3] Baulcombe et al., Plant J., (1995), 7:1045-1053

[Non-Patent Document 4] Christou, Methods Cell Biol., (1995), 50:375-82

### SUMMARY OF THE INVENTION

An aspect of the present invention provides a method for expressing a foreign gene, comprising the steps of:
(a) producing a plant which functionally expresses an RNA1 genome and/or an RNA2 genome of Cucumber Mosaic Virus (CMV);
(b) introducing a nucleic acid vector into the plant, said nucleic acid vector comprising, arranged in this order:

(i) a right border sequence (RB) derived from the Agrobacterium T-DNA sequence;

(ii) an expression promoter sequence workable in the plant;

(iii) a sequence corresponding to an RNA3 genome of CMV in which a 3b gene, which codes for a 3b protein (coat protein) of CMV, has been prevented from being expressed and the foreign gene has been inserted downstream of, and operably linked with, a subgenomic promoter which otherwise promotes the expression of the 3b gene; and

(iv) a left border sequence (LB) derived from the Agrobacterium T-DNA sequence; and
(c) culturing the plant containing the nucleic acid vector such that the RNA1 and/or RNA2 genome(s) of CMV is functionally expressed by the plant while the foreign gene is expressed transiently from the nucleic acid vector along with the CMV RNA3 genome lacking a region corresponding to the 3b gene.

Another aspect of the present invention provides an expression system for expressing a foreign gene, comprising:
(A) a plant which functionally expresses at least either an RNA1 genome or an RNA2 genome of Cucumber Mosaic Virus (CMV); and
(B) a nucleic acid vector comprising, arranged in this order:

(i) a right border sequence (RB) derived from the Agrobacterium T-DNA sequence;

(ii) an expression promoter sequence workable in the plant;

(iii) a sequence corresponding to an RNA3 genome of CMV in which a 3b gene, which codes for a 3b protein (coat protein) of CMV, has been prevented from being expressed and the foreign gene has been inserted downstream of, and operably linked with, a subgenomic promoter which otherwise promotes the expression of the 3b gene; and

(iv) a left border sequence (LB) derived from the Agrobacterium T-DNA sequence.

### BRIEF DESCRIPTION OF THE DRAWINGS

The features and advantages of the present invention will become more apparent from the detailed description set forth below when taken in conjunction with the drawings, wherein:

FIG. 1 schematically illustrates the construction of plasmid pBI-CR1;

FIG. 2 is a photograph indicating the results of genomic PCR analysis on a T1 individual of a CR1-transgenic (Tg) plant, which was obtained by introducing pBI-CR1;

FIG. 3 schematically illustrates the construction of plasmid pBI-CR2;

FIG. 4 schematically illustrates the construction of plasmid pBI-CR3;

FIG. 5 schematically illustrates the constructions of vectors pBI-CR3Δ33delGFP and pBI-CR3Δ33stopGFP;

FIG. 6 includes photographs indicating GFP fluorescence from the CR1-transgenic plant infiltrated with bacterial cells harboring pBI-CR2 and bacterial cells harboring pBI-CR3Δ33delGFP via agroinfiltration;

FIG. 7 is a photograph indicating the results of western blot analysis with an anti-GFP antibody on the CR1-transgenic plant infiltrated with bacterial cells harboring pBI-CR2 and bacterial cells harboring pBI-CR3Δ33delGFP via agroinfiltration;

FIG. 8 schematically illustrates the constructions of vectors pBI-CR3Δ33delGUS and pBI-CR3Δ33stopGUS;

FIG. 9 is a photograph indicating GUS expression from the CR1-transgenic plant infiltrated with bacterial cells harboring pBI-CR2 and bacterial cells harboring pBI-CR3Δ33delGFP via agroinfiltration;

FIG. 10 includes photographs photograph indicating the results of genomic PCR analysis on a T1 individual of a CR2-transgenic plant, which was obtained by introducing pBI-CR2;

FIG. 11 is a photograph indicating GFP fluorescence from the CR2-transgenic plant infiltrated with bacterial cells harboring pBI-CR1 and bacterial cells harboring pBI-CR3Δ33delGFP via agroinfiltration;

FIG. 12 is a photograph indicating GUS expression from CR2-transgenic plant infiltrated with bacterial cells harboring pBI-CR1 and bacterial cells harboring pBI-CR3Δ33delGFP via agroinfiltration;

FIG. 13 includes photograph indicating the results of genomic PCR analysis on a T1 individual of a CR1+CR2-transgenic plant, which was obtained by introducing pBI-CR1 and pBI-CR2;

FIG. 14 includes photographs indicating GFP fluorescence from the CR1+CR2-transgenic plant infiltrated with bacterial cells harboring pBI-CR3Δ33delGFP and bacterial cells harboring pBI-CR3Δ33stopGFP via agroinfiltration; and

FIG. 15 is a photograph indicating GUS expression from the CR1+CR2-transgenic plant infiltrated with bacterial cells harboring pBI-CR3Δ33delGUS via agroinfiltration.

### MODES FOR CARRYING OUT THE INVENTION

The present invention will be explained in detail with reference to specific embodiments. However, the present invention should in no way be limited to the embodiments indicated below, but may be worked with any modifications made as appropriate.

Each of the patent publications, unexamined patent application publications, patent applications, and non-patent documents is incorporated herein by reference in its entirety for all purposes.

### [1. Introduction]

An explanation is first provided of the Agrobacterium and cucumber mosaic virus on which the present invention is premised.

### (1-1. Agrobacterium)

Agrobacterium is the generic name for Gram-negative soil bacteria belonging to the genus Rhizobium that demonstrate pathogenicity against plants. One example of Agrobacterium is Agrobacterium tumefaciens, which is associated with crown gall.

Agrobacterium possesses a giant plasmid referred to as tumor-inducing (Ti) plasmid. The Ti plasmid has a domain referred to as a transfer DNA (T-DNA) sequence, and has the property of being incorporated in a plant genome by random insertion as a result of a DNA fragment corresponding to that domain migrating to a cell in plant. By utilizing this property, Ti plasmid can be used for the purpose of, for example, introducing and expressing a foreign gene in a plant by transformation.

The T-DNA sequence has a right border sequence (to be abbreviated as “RB sequence” or simply “RB”) and a left border sequence (to be abbreviated as “LB sequence” or simply “LB”) on both ends thereof, and has plant hormone biosynthesis genes and opine biosynthesis genes between the RB sequence and LB sequence. The RB and LB sequences are known to play a role not only in incorporation into the plant genome, but also in infection into plant cells and expression of a gene existing between the RB sequence and the LB sequence in plant cells.

Ti plasmid contains sequences essential for plant transformation, including the plant hormone biosynthesis genes and the opine biosynthesis genes mentioned above. The Ti plasmid is therefore quite large, resulting in poor manipulability. Accordingly, introduction of a foreign gene by plant transformation is currently carried out primarily using a T-DNA binary system combining two types of modified Ti plasmids, namely a binary plasmid and a helper plasmid.

The binary plasmid serves as a shuttle vector enabling replication of both Agrobacterium and other microorganisms by incorporating a sequence from which domains not required for plant transformation have been removed from the T-DNA domain (such as the domains of the aforementioned plant hormone biosynthesis genes and opine biosynthesis genes), and a replication origin capable of functioning in both Agrobacterium and other microorganisms (such as Escherichia coli). The binary plasmid normally does not have the entire T-DNA sequence, but rather only has an RB sequence and LB sequence (along with their neighboring domains) derived from the T-DNA sequence, and optionally has a nos promoter and nos terminator, which regulate expression of removed genes such as plant hormone biosynthesis genes and opine biosynthesis genes, between the RB sequence and LB sequence. The foreign gene to be introduced into a plant is arranged between the RB and LB sequences and optionally arranged downstream from the aforementioned nos promoter. In general, a “T-DNA vector” refers to this binary plasmid.

The helper plasmid refers to a plasmid that has been reduced in size by removing the T-DNA sequence and other domains not required for host recombination and so forth of the T-DNA sequence from a Ti plasmid while leaving behind a gene cluster in the form of a vir domain that induces random incorporation of the T-DNA sequence into a genome. The function of the vir domain of inducing recombination of the T-DNA domain into a genome is known to be trans-acting. Accordingly, even if the aforementioned T-DNA vector does not have a vir domain, by incorporating this helper plasmid having a vir domain into Agrobacterium together with the T-DNA vector, the T-DNA domain present in the T-DNA vector can be induced to be incorporated into a genome, and in turn, a foreign gene arranged between the RB sequence and LB sequence can be expressed by incorporating into a plant genome.

Furthermore, a vir domain can be incorporated in the aforementioned T-DNA vector and be made to function as an independent vector without using this type of helper plasmid having a vir domain.

In the case of transiently expressing a foreign gene in a plant by using such a vector derived from Agrobacterium (such as the aforementioned T-DNA vector), the plant is normally infected by introducing the vector into plant tissue by a physical method (see, for example, the aforementioned Non-Patent Document 1: Grimsley, N. et al.). Since vectors derived from Agrobacterium exhibit potent infectious capacity due to the action of the T-DNA domain, if infected by allowing to spread extensively throughout the entire plant by a physical method (such as injection or infiltration), a foreign gene can be uniformly expressed throughout all plant tissue. However, as previously described, since the expression level of the foreign gene in each cell ends up being limited by the abilities of Agrobacterium regulatory sequences (such as nos promoter and nos terminator), it was difficult to improve expression level in the entire plant.

### (1-2. Cucumber Mosaic Virus)

Cucumber mosaic virus (abbreviated as “CMV”) is a virus belonging to the Bromoviridae family that is pathogenic to plants, and has a genome composed of three single-stranded RNA (RNA1, RNA2 and RNA3). RNA1, 2 and 3 have been developed into plasmids in the form of pCY1, pCY2 and pCY3, respectively (Suzuki et al., Virology, Vol. 183, p. 106-113 (1991)).

RNA1 (pCY1) contains a gene that encodes a 1a protein (replicase). RNA2 (pCY2) contains a gene that encodes 2a protein (RNA polymerase) and a gene that encodes 2b protein (silencing suppressor). RNA3 (pCY3) contains a gene that encodes 3a protein (cell-to-cell movement protein) and a gene that encodes a 3b protein (coat protein: CP).

Furthermore, a plasmid vector (C2-H1) has been developed that enables recombination of a foreign gene by introducing a multicloning site into the gene of pCY2 that encodes 2b protein (Matsuo et al., Planta, (2007), 225:277-286). Use of this plasmid makes it possible to transiently express a foreign gene in cells in plant.

According to techniques that use such CMV-derived vectors, the expression level of a foreign gene per cell in plant can be enhanced since the foreign gene is expressed using the high autonomous replication ability of CMV. However, since transmission to each tissue and cell of a plant is dependent upon the transmission ability of the virus, it is extremely difficult to express a foreign gene by infecting the cells of an entire plant with a virus. As a result, following vector introduction, the plant ends up in a chimeric (mosaic) state consisting of cells to which the virus has migrated that express the foreign gene and cells to which the virus has not migrated that do not express the foreign gene. Accordingly, it has remained difficult to improve expression level in an entire plant.

On the other hand, the method described in Patent Document 5 (US2016/0002654A), which was previously filed by the present inventors, involves: preparing a nucleic acid vector containing a sequence corresponding to the RNA2 genome of CMV in which a gene coding for a 2b protein has partially or entirely been replaced with a foreign gene, and a T-DNA sequence of Agrobacterium functionally linked to the sequence corresponding to the CMV RNA2 genome; introducing the nucleic acid molecule into a host plant which functionally expresses the RNA1 and RNA3 genomes of CMV and the 2b protein of CMV; and culturing the plant such that it expresses the foreign gene. This method allows for systemic transition of the foreign gene into cells throughout the plant, with improving the expression efficiency of the foreign gene in each cell, thereby achieving increased expression of the foreign gene in the plant as a whole. This technique also expands the freedom of choice for the type of host plant to be used and the size of target gene to be inserted, compared with the MagnICON® system based on TMV or PVX.

As a result of extensive effort of improving the method described in Patent Document 5, the present inventors have found that the method described in the earlier application requires using a transgenic plant into which the 2b gene was introduced in advance in order to express the foreign gene throughout the plant, i.e., expression of the 2b gene is indispensable. Instead of introducing a foreign gene into the 2b gene of the CMV RNA2 genome as in the method described in the earlier application, the present inventors have conceived of constructing a nucleic acid vector using the CMV RNA3 genome, by preventing expression of a 3b gene, which encodes a 3b protein (CP), while inserting a foreign gene into a region downstream of a subgenomic promoter that controls expression of the 3b gene such that the foreign gene is operably linked with the subgenomic promoter, and then combining the resultant construct functionally with the T-DNA sequences of Agrobacterium. The present inventors have further found that by introducing the resultant nucleic acid vector into a host plant which functionally expresses the RNA1 genome and/or the RNA2 genome of CMV, and then culturing the plant to express the foreign gene, it becomes possible to achieve not only the same effects as those of the method described in the earlier application, but also significant reduction in the time required for expression of the foreign gene (e.g., expression can start two days after inoculation) as well as drastic improvement in the expression amount thereof. Moreover, the present inventors have found that this method allows for expression of a foreign gene of a larger size (e.g., 900 bp or larger), which cannot be achieved by a conventional CMV-based expression system which retains the 3b gene of CMV, such as the method described in the earlier application. Thus, the present inventors have arrived at the present invention.

### [2. Method for Expressing a Foreign Gene]

### (2-1. Summary)

A first aspect of the present invention provides a method for expressing a foreign gene (hereinafter referred to as “the method of the present invention”).

The method of the present invention at least has the following steps:
(a) The step of producing a plant which functionally expresses The CMV RNA1 and/or RNA2 genome(s).
(b) The step of introducing a nucleic acid vector into the plant, said nucleic acid vector comprising, arranged in this order: (i) a right border sequence (RB) derived from the Agrobacterium T-DNA sequence; (ii) an expression promoter sequence workable in the plant; (iii) a sequence corresponding to an RNA3 genome of CMV in which a 3b gene, which codes for a 3b protein (coat protein), of CMV has been prevented from being expressed and the foreign gene has been inserted downstream of, and operably linked with, a subgenomic promoter which otherwise promotes the expression of the 3b gene; and (iv) a left border sequence (LB) derived from the Agrobacterium T-DNA sequence. When the transgenic plant functionally expresses only either the CMV RNA1 or RNA2 genome, then a nucleic acid molecule carrying the CMV RNA1 or RNA2 genome (nucleic acid molecule CR1 or CR2) is also introduced into the plant in order to complement the CMV RNA1 or RNA2 genome, which is not expressed by the transgenic plant.
(c) The step of culturing the plant containing the nucleic acid vector such that the RNA1 and/or RNA2 genome(s) of CMV is functionally expressed by the plant while the foreign gene is expressed transiently from the nucleic acid vector along with the CMV RNA3 genome lacking a region corresponding to the 3b gene. When the nucleic acid molecule CR1 or CR2, which carries the CMV RNA1 or RNA2 genome, is used in combination, then the CMV RNA1 or RNA2 genome is also expressed transiently from the nucleic acid molecule CR1 or CR2.

According to this method, the RNA1 genome and/or the RNA2 genome of CMV, which is functionally expressed by the plant, cooperates with the CMV RNA3 genome lacking the 3b gene, which is transiently expressed from the nucleic acid vector of the present invention, and optionally with the CMV RNA1 or RNA2 genome, which is transiently expressed from the nucleic acid molecule CR1 or CR2 of the present invention, which may optionally be used in some embodiments, to reconstitute the CMV genome products except the 3b gene product and, following infection and proliferation of CMV, result in high expression of the foreign gene included in the nucleic acid vector of the present invention.

Main characteristics of the expression method of the present invention include use of a binary vector constructed by inserting a sequence corresponding to CMV RNA3 between the LB and RB sequences of T-DNA, and replacing the sequence encoding the 3b protein (CP) of CMV with the foreign gene. Conventional virus genome-based binary vectors, including the one described in the earlier patent application by the present inventors, i.e., Patent Document 5 (US2016/0002654A), have restriction on the size of foreign gene that can be introduced and expressed, since they rely on reconstruction of a virus particle encapsulated in the coat protein (according to, e.g., Fukuzawa et al., Plant Biotechnology Journal, (2011), 9:38-49, the maximum size of the gene was 837 bp). On the other hand, the present invention involves using a binary vector in which a foreign gene is inserted into the 3b gene region of the CMV RNA3 genome, which is sandwiched between the LB and RB sequences of T-DNA (the nucleic acid vector of the present invention), and making the resultant vector accumulate in the cytoplasm of plant cells and transiently express a sequence corresponding to CMV RNA3 except the 3b gene, which is complemented with CMV RNA1 and RNA2 (each expressed by a transgenic plant or another nucleic acid molecule) such that the RNA CMV genome products except for the 3b gene are reconstructed. This method allows for expression of the foreign gene by means of the replication ability of CMV even without expression of the coat protein (CP) encoded by the 3b gene. This method also involves no restriction on the size of foreign gene limited by the coat protein (CP), and enables expression of a foreign gene having a larger size (e.g., 900 bp or larger), which was not possible by means of the conventional expression system.

Embodiments of the method of the present invention include, but not limited to, e.g., the following three embodiments.

### Embodiment 1

According to this embodiment, a transgenic plant functionally expressing CMV RNA1 genome (hereinafter referred to as the “CR1-transgenic plant”) is produced in step (a).

Next, in step (b), a nucleic acid vector containing, arranged in this order, (i) a right border sequence (RB) derived from the Agrobacterium T-DNA sequence, (ii) an expression promoter sequence workable in the plant, (iii) a sequence corresponding to the CMV RNA2 genome, and (iv) a left border sequence (LB) derived from the Agrobacterium T-DNA sequence (hereinafter also referred to as “nucleic acid molecule CR2”) is introduced into the CR1-transgenic plant along with the nucleic acid vector of the present invention. In this step, the nucleic acid vector of the present invention and the nucleic acid molecule CR2 should preferably be introduced into the CR1-transgenic plant at the same time by the agroinfiltration method. Specifically, the bacterial suspension of Agrobacterium carrying the nucleic acid vector of the present invention and the bacterial suspension of Agrobacterium carrying the nucleic acid molecule CR2 are prepared, and these bacterial suspensions are mixed and infiltrated or injected into the CR1-transgenic plant.

Then, in step (c), the CR1-transgenic plant into which the nucleic acid vector of the present invention and the nucleic acid molecule CR2 were introduced is cultured such that the CMV RNA1 genome is functionally expressed by the CR1-transgenic plant, while the CMV RNA2 genome carried by the nucleic acid molecule CR2 is transiently expressed in the CR1-transgenic plant, along with foreign gene and the CMV RNA3 genome lacking the region corresponding to the 3b gene carried by the nucleic acid vector.

According to Embodiment 1, the CMV RNA1 genome expressed by the CR1-transgenic plant, the CMV RNA2 genome expressed by the nucleic acid molecule CR2, and the CMV RNA3 genome lacking the region corresponding to the 3b gene expressed by the nucleic acid vector of the present invention cooperate together to reconstitute the CMV RNA genome excluding the 3b gene, and as the CMV infects and proliferates, the foreign gene contained in the nucleic acid vector of the present invention is expressed in a high amount.

### Embodiment 2

According to this embodiment, a transgenic plant functionally expressing CMV RNA2 genome (hereinafter referred to as “CR2-transgenic plant”) is first produced in step (a).

Next, in step (b), a nucleic acid vector containing, arranged in this order, (i) a right border sequence (RB) derived from the Agrobacterium T-DNA sequence, (ii) an expression promoter sequence workable in the plant, (iii) a sequence corresponding to the CMV RNA1 genome, and (iv) a left border sequence (LB) derived from the Agrobacterium T-DNA sequence (hereinafter also referred to as “nucleic acid molecule CR1”) is introduced into the CR2-transgenic plant along with the nucleic acid vector of the present invention. In this step, the nucleic acid vector of the present invention and the nucleic acid molecule CR1 should preferably be introduced into the CR2-transgnic plant at the same time by the agroinfiltration method. Specifically, the bacterial suspension of Agrobacterium carrying the nucleic acid vector of the present invention and the bacterial suspension of Agrobacterium carrying the nucleic acid molecule CR1 are prepared, and these bacterial suspensions are mixed and infiltrated or injected into the CR2-transgenic plant.

Then, in step (c), the CR2-transgnic plant into which the nucleic acid vector of the present invention and the nucleic acid molecule CR1 were introduced is cultured such that the CMV RNA2 genome is functionally expressed by the CR2-transgenic plant, while the CMV RNA1 genome carried by the nucleic acid molecule CR1 is transiently expressed in the CR1-transgenic plant, along with foreign gene and the CMV RNA3 genome lacking the region corresponding to the 3b gene carried by the nucleic acid vector.

According to Embodiment 2, the CMV RNA2 genome expressed by the CR2-transgenic plant, the CMV RNA1 genome expressed by the nucleic acid molecule CR1, and the CMV RNA3 genome lacking the region corresponding to the 3b gene expressed by the nucleic acid vector of the present invention cooperate together to reconstitute the CMV RNA genome excluding the 3b gene, and as the CMV infects and proliferates, the foreign gene contained in the nucleic acid vector of the present invention is expressed in a high amount.

### Embodiment 3

According to this embodiment, a transgenic plant functionally expressing CMV RNA1 genome and RNA2 genome (hereinafter referred to as “CR1+CR2-transgenic plant”) is first produced in step (a).

Next, in step (b), the nucleic acid vector of the present invention is introduced into the CR1+CR2-transgenic plant.

Then, in step (c), the CR1+CR2-itransgenic plant into which the nucleic acid vector of the present invention was introduced is cultured such that the CMV RNA1 and RNA2 genomes are functionally expressed by the CR1+CR2-transgenic plant, while the foreign gene and the CMV RNA3 genome lacking the region corresponding to the 3b gene carried by the nucleic acid vector are transiently expressed in the CR1+CR2-transgenic plant.

According to Embodiment 3, the CMV RNA1 and RNA2 genomes expressed by the CR1+CR2-transgenic plant and the CMV RNA3 genome lacking the region corresponding to the 3b gene expressed by the nucleic acid vector of the present invention cooperate together to reconstitute the CMV RNA genome excluding the 3b gene, and as the CMV infects and proliferates, the foreign gene contained in the nucleic acid vector of the present invention is expressed in a high amount.

The method of the present invention will then be explained in more details.

### (2-2. Transgenic Plant)

Examples of transgenic plants used in the method of the present invention include: a CR1-transgenic plant functionally expressing CMV RNA1 genome (used in Embodiment 1), a CR2-transgenic plant functionally expressing CMV RNA2 genome (used in Embodiment 1), and a CR1+CR2-transgenic plant functionally expressing both CMV RNA1 and RNA2 genomes (used in Embodiment 3).

There are no particular limitations on the transgenic plant used in the method of the present invention, and any arbitrary species of plant can be used. Examples of plants to which the present invention can be applied include alfalfa, barley, kidney beans, canola, cowpeas, cotton, corn, clover, lotus flower, lentil, lupine, sugar cane, oats, peas, peanuts, rice, rye, sweet clover, sunflower, sweet peas, soybeans, sorghum, triticale, yam beans, velvet beans, broad beans, wheat, wisteria and nut plants.

Preferable examples include gramineous plants, asteraceae plants, foliaceous plants and rosaceae plants.

More preferable examples of plants include plants belonging to the following genii: Arabidopsis, Agrostis, Allium, Antirrhinum, Apium, Arachis, Asparagus, Atropa, Avena, Bambusa, Brassica, Bromus, Browallia, Camellia, Cannabis, Capsicum, Cicer, Chenopodium, Cichorium, Citrus, Coffea, Coix, Cucumis, Cucurbita, Cynodon, Dactylis, Datura, Dacus, Digitalis, Dioscorea, Elaeis, Eleusine, Festuca, Fragaria, Geranium, Glycine, Helianthus, Hemerocallis, Hevea, Hordeum, Hyoscyamus, Ipomoea, Lactuca, Lens, Lilium, Linum, Lolium, Nelumbo, Lycopersicon, Majorana, Malus, Mangifera, Manihot, Medicago, Nemesia, Nicotiana, Onobrychis, Oryza, Panicum, Pelargonium, Pennisetum, Petunia, Pisum, Phaseolus, Phleum, Poa, Prunus, Ranunculus, Raphanus, Ribes, Ricinus, Rubus, Saccharum, Salpiglossis, Secale, Senecio, Setaria, Sinapis, Solanum, Sorghum, Stenotaphrum, Theobroma, Trifolium, Trigonella, Triticum, Vicia, Vigna, Vitis and Zea.

In addition to a whole plant, a culture of a partial tissue or cells of such a plant can be used.

The transgenic plant used in the method of the present invention is recombined such as to functionally express CMV RNA1 genome and/or RNA2 genome. Such a transgenic plant can be prepared by, e.g., introducing a sequence corresponding to the CMV RNA1 genome and/or a sequence corresponding to the CMV RNA2 genome into a subject plant and incorporating it into the plant genome such that it is functionally expressed. There are no limitations on the timing or order in which these sequences are introduced into the target plant. Namely, these sequences may be introduced simultaneously or may be introduced separately in any arbitrary order.

Typical methods for introducing a sequence corresponding to the CMV RNA1 genome and/or a sequence corresponding to the CMV RNA2 genome into a plant include transformation methods using plant expression vectors, which may be any arbitrary vectors. Examples include various virus vectors, phage vectors, plasmid vectors, and binary vectors. Preferable among these methods are methods using binary vectors in which a plasmid carrying a sequence corresponding the CMV RNA1 or RNA2 genome has been incorporated into Agrobacterium, and recombining a plant with the vector by the leaf disc method. Detailed conditions for plant expression vectors and transformation may be selected as appropriate from known conditions, depending on the subject plant and the sequence to be introduced. For details of the leaf disc method, reference can be made to, e.g., Horsch et al., Science, (1984), 223:496-498.

The following explanation will be made into further details of a method of using a binary vector into which a plasmid carrying a sequence corresponding to the CMV RNA1 or RNA2 genome has been incorporated into Agrobacterium, and recombining the plant with the vector by the leaf disc method.

Binary vectors in which a plasmid a sequence corresponding to the CMV RNA1 or RNA2 genome has been incorporated into Agrobacterium may have similar constructions to that of the nucleic acid molecule R1 or R2 of the present invention (hereinafter may be referred to as binary vectors R1 and R2). However, unlike the nucleic acid molecule R1 or R2 of the present invention, since the binary vectors R1 and R2 are used in the production of a transgenic plant, they should not be transiently expressed in the plant cytoplasm, but must be incorporated into the plant genome. For this purpose, a sequence essential for incorporation into the plant genome, e.g., the vir region derived from the Ti plasmid of Agrobacterium, must be inserted into the binary vector R1 or R2 or, alternatively, a helper plasmid containing the vir region must be used in combination.

It is also preferred to use a selection marker gene in order to confirm incorporation of the CMV RNA1 or RNA2 sequence into the plant genome. Examples of selection marker genes are not limited, but typically include various antibiotic resistance genes and medicament resistance genes, e.g., genes conferring resistance to ampicillin, streptomycin, gentamicin, kanamycin, hygromycin, actinonin (PDF1 gene), bialaphos herbicide, glyphosate herbicide, sulfonamide, or mannose. Such a selection marker gene is typically operably linked to control sequences such as an inherent promoter to form an expression cassette which can autonomously express in the plant genome (hereinafter also referred to as “selection marker expression cassette”), and the expression cassette is disposed between (a) the right border sequence (RB) and (e) the left border sequence (LB) (on the 3′ side or on the 5′ side of the expression cassette of the present invention). Thus, when the right border sequence (RB) and the left border sequence (LB) cause random incorporation into the genome, the selection marker expression cassette (along with the expression cassette of the present invention) is introduced into the plant genome and thereby expresses autonomously.

A transgenic plant functionally expressing a plurality of products (i.e., a CR1+CR2transgenc plant CMV functionally expressing both the RNA1 genome and the RNA2 genome) can be produced by using both binary vectors R1 and R2 and recombining the same plant with these vectors by the leaf disc method. Alternatively, such a transgenic plant can be produced by preparing transgenic plants functionally expressing individual products (e.g., a CR1-transgenic plant functionally expressing CMV RNA1 genome and a CR2-transgenic plant functionally expressing CMV RNA2 genome), and then crossbreeding these transgenic plants, followed by screening of the resultant individuals for a desired transgenic plant.

Screening produced transgenic plants for a desired transgenic plant functionally expressing the CMV RNA1 and/or RNA2 genome(s) can be carried out by, e.g., preparing a binary vector carrying each of the CMV RNA genome(s) not functionally expressed by the transgenic plant (i.e., CMV RNA2 and RNA3 in the case of a CR1-transgenic plant, CMV RNA1 and RNA3 in the case of a CR2-transgenic plant, and CMV RNA3 in the case of a CR1+CR2-transgenic plant) between the LB and RB sequences of T-DNA, then introducing the vector into the transgenic plant by, e.g., the leaf disc method such that it is transiently expressed in the plant, and confirming as to whether the reconstruction of the CMV RNA genomes and the resultant infection of CMV into the plant are observed. If infection of CMV into the transgenic plant is observed, then it is confirmed that the transgenic plant functionally expresses the desired CMV RNA genome(s). The Examples below can be referred to for details of the screening method.

The transgenic plants functionally expressing the CMV RNA1 genome and/or the CMV RNA2 genome mentioned above (i.e., the CR1-transgenic plant, the CR2-transgenic plant, and the CR1+CR2-transgenic plant) are also included in the subject of the present invention. These transgenic plants may also be collectively referred to as “the transgenic plant of the present invention”.

### (2-3. Foreign Gene)

The foreign gene used in the method of the present invention are not limited, but may be any arbitrary gene. Examples include a variety of naturally-occurring genes and synthetic genes, as well as fragments, variants, and modified genes thereof.

The length of the foreign gene is also not limited. However, like the method described in Patent Document 5 (US2016/0002654A), which is an earlier patent application by the present inventors, the conventional CMV-based expression systems retaining the 3b gene of CMV coat protein (CP) have restrictions on the size of foreign gene which can be expressed (e.g., according to Fukuzawa et al., Plant Biotechnology Journal (2011), 9:38-49, the maximum gene size was 837bp). On the other hand, the present invention has no restrictions on the size of foreign gene due to CP, and allows for introduction of longer genes (e.g., 900 bp or larger, particularly 1000 bp or larger) into the nucleic acid vector and have them expressed in the plant. Therefore, the present invention is especially useful for causing expression of long-chain genes.

There are no restrictions on the type of the foreign gene, which may be any arbitrary gene. Specific examples include, but not limited to, various cytokines, immunogens, antibodies, enzymes, blood-derived ingredients, adjuvant-active ingredients, virus-derived ingredient, and pathogenic microorganism-derived ingredients.

### (2-4. Nucleic Acid Vector)

The nucleic acid vector used in the method of the present invention contains the following sequences (a) to (e), arranged in this order, of which the sequence (d) is optional.
(a) A right border sequence (RB) derived from the Agrobacterium T-DNA sequence.
(b) An expression promoter sequence workable in the subject plant.
(c) a sequence corresponding to an RNA3 genome of CMV in which the gene coding for a 3b protein (CP) of CMV has been prevented from being expressed and the foreign gene has been inserted downstream of, and operably linked with, a subgenomic promoter which otherwise promotes the expression of the gene coding for the 3b protein (hereinafter also referred to as “the foreign gene-containing CMV RNA3 corresponding sequence”).
(d) A terminator sequence workable in the subject plant.
(e) A left border sequence (LB) derived from the Agrobacterium T-DNA sequence.

The sequences of (a) and (e) above, i.e., the right border sequence (RB) and the left border sequence (LB) both derived from the Agrobacterium T-DNA sequence, are already explained above.

The expression promoter sequence of (b) above is not limited as long as it can function in the subject plant genome and initiate transcription of the coding sequence of the foreign gene. However, in order to express the foreign gene in the subject plant with high efficiency, it is preferred to use a promoter having stronger activity than the Agrobacterium-derived nos promoter. Examples of such promoters include cauliflower mosaic virus (CaMV) 35S RNA promoter (Odell et al., (1985), Nature, 313:810-812), cassava mosaic virus promoter, scrophularia mosaic virus promoter, badnavirus promoter, strawberry vein banding virus (SVBV) promoter, mirabilis mosaic virus promoter (MMV), rubisco promoter, actin promoter, and ubiquitin promoter. Especially preferable is a promoter derived from 35S RNA of cauliflower mosaic virus (CaMV).

The sequence corresponding to the CMV RNA3 genome used in the foreign gene-containing CMV RNA3 corresponding sequence of (c) above may typically be a cDNA sequence of the RNA3 genome. As mentioned above, the CMV RNA3 genome contains a 3a gene, which encodes a 3a protein, and a 3b gene, which encodes a 3b protein (coat protein: CP). In the present invention, the cDNA sequence of the RNA3 genome is modified such that expression of a partial sequence corresponding to the 3b gene is inhibited. Specifically, the 3b gene should preferably be deleted by replacing the sequence corresponding to the 3b gene with a foreign gene.

The mode of replacement is not limited. However, from the viewpoint of improving the transcription efficiency of the foreign gene, the start codon of the coding sequence of the foreign gene should be placed downstream of, and operably linked to, the subgenomic promoter for expressing RNA3b. The length of the foreign gene is not limited, and may be longer or shorter than the length of the sequence to be replaced, as long as incorporation of the RNA3 genome into a CMV particle is not inhibited. In other words, the length of the gene may not necessarily be the same before and after the replacement. As mentioned above, the present invention allows for incorporation of a longer gene (e.g., 900 bp or larger, particularly 1000 bp or larger) into the nucleic acid vector, and can cause expression of such a longer gene in the plant. Accordingly, the present invention is especially useful when expressing a long-chain gene.

For the purpose of, e.g., improving the transcription/translation efficiency of the virus, various modifications can be made to the sequence. Preferred examples of such modifications include: introduction of a certain sequence (e.g., “A/TXXAUGXC”: where X means an arbitrary base) in the vicinity of the start codon (AUG), introduction of an internal ribosomal entry site (IRES) sequence, and introduction of a ribozyme-cleavable sequence.

The terminator sequence of (d) above is an optional component, as mentioned above. However, from the viewpoint of surely terminating transcription of the coding sequence of the foreign gene and causing expression of a desired functional protein, the nucleic acid vector of the present invention should preferably contain a terminator sequence. The specific terminator sequence is not limited, as long as it can terminate transcription of the coding sequence of the foreign gene. Examples include Agrobacterium-derived nos terminator, heat shock protein (hsp) terminator, and cauliflower mosaic virus (CaMV) 35S RNA terminator.

It would be acknowledged that (b) the expression promoter sequence and (c) the foreign gene-containing CMV RNA3 corresponding sequence (as well as (d) the optional terminator sequence) should be operably linked to each other such that the RNA3 genome lacking the CMV 3b gene and foreign gene included in the sequence of (c) above can be expressed. Namely, the sequences of (b) and (c) above (as well as the optional sequence of (d)) should constitute an expression cassette which can autonomously express in the plant genome (this expression cassette is hereinafter also referred to as “the expression cassette of the present invention”). In other words, the expression cassette of the present invention can be introduced into the plant genome and autonomously express the 3a protein and the foreign gene.

The foreign gene-containing CMV RNA3 corresponding sequence included in the nucleic acid vector of the present invention is not limited, as long as it is a sequence corresponding to the CMV RNA3 genome in which expression of the 3b gene is inhibited, and the foreign gene has been inserted to downstream of the subgenomic promoter controlling expression of the 3b gene, and is operably linked to the subgenomic promoter. However, the foreign gene-containing CMV RNA3 corresponding sequence should at least contain sequences on the CMV RNA3 genome which are necessary for CMV virus proliferation, i.e., the 5-prime sequence, which is located upstream (on the 5′ side) of the coding region of the 3a gene of CMV RNA3, a region located between the 3a gene and the 3b gene including the subgenomic promoter, and the 3-prime sequence, which is located upstream (on the 3′ side) of the coding region of the 3b gene of CMV RNA3. This construction allows for transient expression of the CMV RNA3 genome lacking the region corresponding to the 3b gene in the plant, thereby efficiently causing reconstruction of CMV by necessary proteins except CP.

In this connection, a specific region of CMV RNA3 on the C-terminal side of the 3a protein is known to interact with expression of the 3b protein (CP) at the protein level. Inhibiting expression of the specific region of CMV RNA3 on the C-terminal side of the 3a protein allows for cell-to-cell movement without depending on CP. Therefore, the nucleic acid vector of the present invention should preferably be constructed such that in the foreign gene-containing CMV RNA3 corresponding sequence, the specific region of CMV RNA3 on the C-terminal side of the 3a protein that can interact with expression of the 3b protein (CP) is prevented from expressing. Such a nucleic acid vector can be used to avoid expression of the region of the 3a protein on the C-terminal side in addition to expression of the 3b protein (CP), thereby saving the energy necessary for producing the 3a protein upon virus proliferation, and causing quick shift to production of the target protein with efficiency.

The region of CMV RNA3 on the C-terminal side of the 3a protein interacting with expression of the 3b protein (CP) may defer depending on the CMV strains. However, it has been reported that for RNA3 of CMV-R strain, for example, the region consisting of 33 amino acids from the C-terminal of the 3a protein can interact with the 3b protein (see, e.g., Kim S. H., Journal of General Virology, (2004), 85:221-230; and Hwang et al. BMC Biotechnology (2012), 12:66). Therefore, it is preferred to inhibit expression of a region generally corresponding to this reported region. Specifically, expression of a region consisting of 35 amino acids, or 34 amino acids, or 33 amino acids, or 32 amino acids, or 31 amino acids on the C-terminal side of the 3a protein should preferably be inhibited.

Inhibition of expression of the region of the 3a protein on the C-terminal side expression can be achieved by, e.g., deleting a 3′-side region of the 3a gene corresponding to the region on the C-terminal side, or by inserting a stop codon to upstream of the 3′-side region of the 3a gene. However, the latter method of stop codon insertion is preferred, since it allows for expression of a full-length mRNA corresponding to the 3a gene at the transcription stage while deleting the specific region of the 3a protein on the C-terminal side at the translation stage, thereby maximizing the expression efficiency.

The nucleic acid vector of the present invention may contain any other sequence as long as it does not substantially prevent the function of the nucleic acid vector.

The nucleic acid vector of the present invention may be either in a liner form or in a cyclic form. However, it should preferably be in a cyclic form, e.g., a plasmid form, and should more preferably be in the form of a T-DNA vector, which has replication ability in Agrobacterium.

The nucleic acid vector of the present invention having a structure as mentioned above can easily be prepared by selecting and combining various gene recombination techniques well-known to persons skilled in the art as appropriate.

As mentioned above, the nucleic acid vector of the present invention has a construction containing, between the RB and LB sequences of the Agrobacterium-derived T-DNA sequence, an expression cassette which contains the sequence corresponding to CMV RNA3, in which the foreign gene has been inserted in place of the 3b gene, is operably linked to an expression promoter (the expression cassette of the present invention). The nucleic acid vector of the present invention is distributed throughout the plant by a physical means mentioned below (e.g., injection or infiltration) to cause infection. As a result, the nucleic acid molecule is accumulated in the cytoplasm of each plant cell with high efficiency, and can transiently express a CMV RNA3 genome product lacking the 3b gene in the plant.

### (2-5. Nucleic Acid Molecule)

According to the method of the present invention, in addition to the nucleic acid vector mentioned above, the nucleic acid molecule CR1 or CR2 explained below (hereinafter also collectively referred to as “the nucleic acid molecule of the present invention”) is used depending on embodiments and modifications.

The nucleic acid molecule CR1 contains the sequences (a), (b), (c′), (d) and (e) explained below, arranged in this order. The sequence of (d) below is optional.
(a) A right border sequence (RB) derived from the Agrobacterium T-DNA sequence.
(b) An expression promoter sequence workable in the subject plant.
(c′) A sequence corresponding to the CMV RNA1 genome.
(d) A terminator sequence workable in the subject plant.
(e) A left border sequence (LB) derived from the Agrobacterium T-DNA sequence.

The sequences of (a), (b), (d) and (e) below were already explained in relation to the nucleic acid vector of the present invention.

The sequence (c′), i.e., a sequence corresponding to the CMV RNA1 genome, may typically be a cDNA sequence of the CMV RNA1 genome. This sequence may include various modifications for the purpose of, e.g., improving the transcription/translation efficiency of the virus. Preferred examples of such modifications are those explained above for the sequence (c) of the nucleic acid molecule of the present invention.

In the nucleic acid molecule CR1, (b) the expression promoter sequence and (c′) the sequence corresponding to the CMV RNA1 genome (as well as (d) the optional terminator sequence) should be operably linked to each other such that the sequence corresponding to the CMV RNA1 genome of (c′) above can be expressed, i.e., should constitute an expression cassette which can autonomously express in the plant genome (this expression cassette is hereinafter also referred to as “the expression cassette A”).

The nucleic acid molecule CR2 contains the sequences (a), (b), (c″), (d) and (e) explained below, arranged in this order. The sequence of (d) below is optional.
(a) A right border sequence (RB) derived from the Agrobacterium T-DNA sequence.
(b) An expression promoter sequence workable in the subject plant.
(c′) A sequence corresponding to the CMV RNA2 genome.
(d) A terminator sequence workable in the subject plant.
(e) A left border sequence (LB) derived from the Agrobacterium T-DNA sequence.

The sequences of (a), (b), (d) and (e) below were already explained in relation to the nucleic acid vector of the present invention.

The sequence (c″), i.e., a sequence corresponding to the CMV RNA2 genome, may typically be a cDNA sequence of the CMV RNA1 genome. This sequence may include various modifications for the purpose of, e.g., improving the transcription/translation efficiency of the virus. Preferred examples of such modifications are those explained above for the sequence (c) of the nucleic acid molecule of the present invention.

In the nucleic acid molecule CR1, (b) the expression promoter sequence and (c″) the sequence corresponding to the CMV RNA2 genome (as well as (d) the optional terminator sequence) should be operably linked to each other such that the sequence corresponding to the CMV RNA2 genome of (c″) above can be expressed, i.e., should constitute an expression cassette which can autonomously express in the plant genome (this expression cassette is hereinafter also referred to as “the expression cassette B”).

The nucleic acid molecules CR1 and CR2 may contain any other sequence as long as it does not substantially prevent the function of the nucleic acid molecules.

The nucleic acid molecules CR1 and CR2 may be either in a liner form or in a cyclic form. However, they should preferably be in a cyclic form, e.g., a plasmid form, and should more preferably be in the form of a T-DNA vector, which has replication ability in Agrobacterium.

As mentioned above, the nucleic acid molecules CR1 and CR2 each have a construction containing, between the RB and LB sequences of the Agrobacterium-derived T-DNA sequence, an expression cassette which contains the sequence corresponding to CMV RNA1 or RNA2, respectively, is operably linked to an expression promoter. Each of the nucleic acid molecules CR1 and CR2 is distributed throughout the plant by a physical means mentioned below (e.g., injection or infiltration) to cause infection. As a result, the nucleic acid molecule is accumulated in the cytoplasm of each plant cell with high efficiency, and can transiently express a CMV RNA1 or RNA2 genome product.

(2-6. Introduction of the Nucleic Acid Vector/Nucleic Acid Molecules into the Transgenic Plant)

In the method of the present invention, the transgenic plant is produced in step (a), and the nucleic acid vector of the present invention is introduced into the plant in step (b), along with the nucleic acid molecule CR1 or CR2 depending on the embodiments. The method for introducing the nucleic acid vector and the nucleic acid molecules into the transgenic plant is not limited, but should preferably include, e.g., preparing a solution containing the nucleic acid vector or the nucleic acid molecule of the present invention, and introducing the solution into the plant tissues by various physical means such as injection and infiltration.

The solution containing the nucleic acid vector or the nucleic acid molecule of the present invention can be prepared by, e.g., culturing a microorganism such as Agrobacterium that produces the nucleic acid vector of the present invention, and then using the resulting culture (such as an overnight culture). Normally, an overnight culture reaches an optical density at a wavelength of 600 nm (OD₆₀₀) of 3 to 3.5 units. This culture is then diluted 3 to 5 times prior to agroinfiltration to typically form 5×10⁹ to 9×10⁹ colony forming units (Turpen, et al., J. Virol. Methods, (1993), 42:227-240). The resulting culture is then diluted 10²-fold, preferably 10³-fold and more preferably 10⁴-fold using a buffer such as MES buffer to suspend at an OD₆₀₀ value of 0.2 to 0.8 units. In the case of using each of these microbial solutions alone, suspensions are prepared at an OD₆₀₀ value of 0.2 to 0.6 units. In the case of using a mixture of a plurality of these microbial solutions, equal amounts of each of the bacterial cells are mixed followed by adjusting the final amount of the bacterial cells in the microbial solutions to an OD₆₀₀ value of 0.3 to 1.2 units.

In the case of introducing the solution containing the nucleic acid vector or the nucleic acid molecule of the present invention into a target plant by injecting a solution thereof, introduction is carried out by forcibly injecting the solution into the target plant using a syringe and the like. In the case of introducing a solution containing the nucleic acid molecule of the present invention into a target plant by infiltration, introduction is carried out by first allowing the solution containing the nucleic acid vector or the nucleic acid molecule of the present invention to contact the target plant followed by forcibly causing the solution to infiltrate the target plant under reduced pressure conditions using, for example, a vacuum desiccator.

These means are used to allow the nucleic acid vector or the nucleic acid molecule of the present invention to a part or all of the tissues of the target plant. It is preferred to allow the nucleic acid vector or the nucleic acid molecule of the present invention to uniformly spread throughout all tissue of the target plant. Thus, the nucleic acid vector of the present invention introduced into the plant, along with the nucleic acid molecule CR1 or CR2 optionally used, is accumulated in the cytoplasm of the plant cells with high efficiency, and is able to transiently express its CMV genome-corresponding sequence.

The transgenic plant into which the nucleic acid vector of the present invention (and the nucleic acid molecule CR1 or CR2, which is optionally used depending on the embodiments) was introduced in step (b) is then cultured in step (c) of the method of the present invention. The method used to cultivate the target plant may be suitably selected corresponding to the type of target plant and purpose of expressing the foreign gene. Thus, the CMV RNA1 genome and/or the RNA2 genome is/are functionally expressed by the transgenic plant, and the CMV RNA3 genome lacking the 3b gene is transiently expressed by the nucleic acid vector of the present invention accumulated in the cytoplasm of the infiltrated transgenic plant. In addition, when the nucleic acid molecule CR1 or CR2 was introduced into the transgenic plant depending on the embodiments, the CMV RNA1 or the RNA2 genome is transiently expressed by the nucleic acid molecule CR1 or CR2, respectively, accumulated in the cytoplasm of the infiltrated transgenic plant. The thus-expressed CMV RNA1 genome, RNA2 genome, and CMV RNA3 genome lacking the 3b gene cooperate with each other to reconstruct the genome product of CMV lacking the 3b gene product, and as the CMV infects and proliferates, the foreign gene included in the nucleic acid vector of the present invention is highly expressed.

### (2-7. Summary)

According to the method of the present invention explained above, a transgenic plant functionally expressing the RNA1 genome and/or the RNA2 genome of CMV is infiltrated by the nucleic acid vector of the present invention, optionally with the nucleic acid molecule CR1 or CR2 as appropriate, to express the CMV genome expression products except the 3b gene product and make them complement each other in trans. The foreign gene can then be expressed due to the high autonomous replication ability of CMV. This method can thus allow the foreign gene to uniformly spread the cells throughout the plant and make it express, and also can improve the expression efficiency of the foreign gene in each cell, thereby achieving high level of expression from the plant as a whole. This technique also expands the freedom of choice for the type of host plant to be used.

In addition, the expression method of the present invention has no restriction on the size of foreign gene, since the coat protein (CP) of CMV is not expressed. Thus, this method allows for expression of a foreign gene of a larger size (e.g., 900 bp or larger), which cannot be achieved by a conventional magnICON® system, or even by the previous method made by the present inventors as described in the earlier application, i.e., Patent Document 5 (US2016/0002654A).

The method of the present invention can also prevent diffusion of the foreign gene or CMV. Namely, in the method of the present invention, the CMV RNA1 genome and/or the CMV RNA2 genome is/are provided via stable expression by the transgenic plant or, otherwise, by the nucleic acid molecule CR2 or CR1, respectively, introduced transiently into the transgenic plant. Accordingly, reconstruction of the CMV genomes only occurs inside the infiltrated transgenic plant. In addition, the CMV RNA3 genome provided by the nucleic acid vector of the present invention lacks CP, which is indispensable for the long-range movement of the virus. Therefore, no CMV virus particles are formed in the infiltrated transgenic plant, whereby the foreign gene or the CMV carrying the foreign gene is prevented from leaking outside the infiltrated transgenic plant is prevented. In addition, CMV is known to be borne by aphid, and aphid-borne transmission of CMV requires CP. Accordingly, the method of the present invention can also prevent diffusion of the CMV vector carrying the foreign gene via aphid-borne transmission, since the CMV genome products lack CP.

It should also be noted that a binary vector similar to the nucleic acid vector of the present invention was known, i.e., a vector containing a sequence corresponding to CMV RNA3, in which the 3b gene is replaced with the foreign gene and which is sandwiched between the T-DNA RB and LB sequences of Agrobacterium (see, e.g., Sudarshana et al., Plant Biotechnology Journal (2006) 4, pp. 551-559). However, there has been no prior art disclosing the technical concept of causing such a vector to infect a transgenic plant functionally expressing the CMV RNA1 and/or RNA2 genome(s), optionally in combination with a binary vector having the CMV RNA1 or RNA2 genome (i.e., a vector corresponding to the nucleic acid molecule CR1 or CR2), to cause expression of the CMV genome products except the 3b gene product and complement with each other in trans, thereby also causing transient expression of the foreign gene by means of the replication ability of CMV. Nor has there been any prior art disclosing various advantages achieved by this method, i.e., improvement in the expression efficiency of the foreign gene, increase in the size of foreign gene that can be expressed, or prevention of the foreign gene from diffusing outside the transgenic plant.

### [3. Expression System for Expressing a Foreign Gene]

The expression system for achieving the method of the present invention mentioned above is also included in the subject of the present invention. Namely, the expression system of the present invention is expression system for expressing a foreign gene, comprising: (A) a plant which functionally expresses at least either an RNA1 genome or an RNA2 genome of CMV; and (B) a nucleic acid vector comprising, arranged in this order: (i) a right border sequence (RB) derived from the Agrobacterium T-DNA sequence; (ii) an expression promoter sequence workable in the plant; (iii) a sequence corresponding to an RNA3 genome of CMV in which the gene coding for a 3b protein (CP) of CMV has been prevented from being expressed and the foreign gene has been inserted downstream of, and operably linked with, a subgenomic promoter which otherwise promotes the expression of the gene coding for the 3b protein of CMV; and (iv) a left border sequence (LB) derived from the Agrobacterium T-DNA sequence.

The details of the transgenic plant of the present invention and the nucleic acid vector of the present invention were already explained in [2. Method for expressing a foreign gene] above. Embodiments of the expression system of the present invention include, but not limited to, the following:

### Embodiment 1

The expression system of this embodiment includes a CR1-transgenic plant, which has been recombined such as to functionally express the CMV RNA1 genome, as the infiltrated transgenic plant of the present invention of (A) above. The expression system of this embodiment also includes (C) a nucleic acid molecule CR2 comprising, arranged in this order: (i) a right border sequence (RB) derived from the Agrobacterium T-DNA sequence; (ii) an expression promoter sequence workable in the plant; (iii) a sequence corresponding to the CMV RNA2 genome; and (iv) a left border sequence (LB) derived from the Agrobacterium T-DNA sequence. The expression system of Embodiment 1 can be used for carrying out the Embodiment 1 of the method of the present invention mentioned above. The details of the CR1-transgenic plant and the nucleic acid molecule CR2 were already explained in [2. Method for expressing a foreign gene] above.

### Embodiment 2

The expression system of this embodiment includes a CR2-transgenic plant, which has been recombined such as to functionally express the CMV RNA2 genome, as the infiltrated transgenic plant of the present invention of (A) above. The expression system of this embodiment also includes (C) a nucleic acid molecule CR1 comprising, arranged in this order: (i) a right border sequence (RB) derived from the Agrobacterium T-DNA sequence; (ii) an expression promoter sequence workable in the plant; (iii) a sequence corresponding to the CMV RNA1 genome; and (iv) a left border sequence (LB) derived from the Agrobacterium T-DNA sequence. The expression system of Embodiment 2 can be used for carrying out the Embodiment 2 of the method of the present invention mentioned above. The details of the CR2-transgenic plant and the nucleic acid molecule CR1 were already explained in [2. Method for expressing a foreign gene] above.

### Embodiment 3

The expression system of this embodiment includes a CR1+CR2-transgenic plant, which has been recombined such as to functionally express a CMV RNA1 genome and a CMV RNA2 genome, as the infiltrated transgenic plant of the present invention of (A) above. The expression system of Embodiment 3 can be used for carrying out the Embodiment 3 of the method of the present invention mentioned above. The details of the CR1+CR2-transgenic plant were already explained in [2. Method for expressing a foreign gene] above.

### [4. Others]

As mentioned above, according to the expression method and system of the present invention, a transgenic plant which can permanently express the full-length segment(s) of the CMV RNA1 and/or RNA2 genome(s) is produced, and then infiltrated with the nucleic acid vector of the present invention which can transiently express the CMV RNA3 genome lacking the 3b gene sequence and, when the transgenic plant expresses the segment of only either the CMV RNA1 or RNA2 genome, also with the nucleic acid molecule of the present invention which can transiently express the remaining CMV segment genome, via Agrobacterium to complement the CMV segment genomes, thereby inducing the proliferation of CMV and the following expression of the foreign gene.

The expression method and system of the present invention allow for significant reduction in the time required for expression of the foreign gene (e.g., expression can start two days after inoculation) as well as drastic improvement in the expression amount thereof. The method and system also allow for expression of a foreign gene of a larger size (e.g., 900 bp or larger), which cannot be achieved by a conventional CMV-based expression system which retains the CMV 3b gene, such as the method described in the earlier application. They also can facilitate the operations, achieve high efficiency expression, and prevent diffusion of the virus.

Therefore, the expression method and system of the present invention is useful in, although not limited to, production of pharmaceutical drugs for emergency use, such as therapeutic proteins, vaccines, and antibodies.

Specifically, examples of therapeutic proteins that can be produced by the expression method and system of the present invention include: enzymes used as recombinant pharmaceuticals (t-PA, glucocerebrosidase, N-acetylgalactosamine-6-sulfatase, α-galactosidase, α-L-iduronidase, acidic α-glucosidase, iduronate-2-sulfatase, N-acetylgalactosamine-4-sulfatase, urate oxidase, DNase, alkaline phosphatase+Fc, collagenase, lysosomal acid lipase, etc.); serum proteins (albumin, etc.); blood coagulation/fibrinolysis-associated factors (blood coagulation factor VII, blood coagulation factor VIII, blood coagulation factor VIII analogs, blood coagulation factor IX, blood coagulation factor XIII, thrombomodulin, antithrombin, etc.); hormones (insulin, insulin analogs, growth hormones, growth hormone analogs, somatomedin C, natriuretic peptides, glucagon, follicle-stimulating hormone, chorionic gonadotropin, GLP-1 analogs, parathyroid hormone analogs, leptin, etc.), interferons (interferon α, interferon β, interferon γ, etc.), erythropoietins (erythropoietin, erythropoietin analogs, etc.), cytokines (G-CSF, G-CSF derivative, interleukin-2, bFGF, etc.), functional peptides (soluble TNFR, CTLA4, TPOR antagonist peptide fusion protein, VEGFR), and other functional proteins.

Examples of vaccines that can be produced by the expression method and system of the present invention include: hepatitis B vaccines, hepatitis A vaccines, vaccines to prevent HPV infection, and influenza vaccines.

Examples of antibodies that can be produced by the expression method and system of the present invention include: various therapeutic antibodies such as mouse antibodies, chimeric antibodies, humanized antibodies, human antibodies. Specifically, mouse anti-CD3 antibody, humanized anti-HER2 antibody, chimeric anti-CD20 antibody, humanized anti-RS virus antibody, chimeric anti-TNFα antibody, chimeric anti-CD25 antibody, humanized anti-IL6Rantibody, humanized anti-CD3 antibody, humanized anti-VEGF antibody, mouse anti-CD20 antibody, human anti-TNFα antibody, chimeric anti-EGFR antibody, humanized anti-IgE antibody, human anti-complement C5 antibody, human anti-EGFR antibody, human anti-IL2/IL23-p40 antibody, humanized anti-TNFα antibody, human anti-IL-1β antibody, human anti-RANKL antibody, humanized anti-CCR4 antibody, human anti-CD20 antibody, humanized anti-HER2 antibody, chimeric anti-CD30 antibody, humanized anti-α4 integrin antibody, human anti-PD-1 antibody, humanized anti-CD52 antibody, human anti-IL-17Aantibody, human anti-VEGF-2 antibody, humanized anti-CTLA-4 antibody, human anti-PCSK9 antibody, humanized anti-IL-5 antibody, humanized anti-IL-17 antibody, humanized anti-IL-17Rantibody, humanized anti-dabigatran antibody, humanized anti-SLAMF7 antibody, and humanized anti-PD-1 antibody.

The expression method and system of the present invention can also be used for the production of an IgG antibody, which is used in, e.g., a reagent, and for the production of an associated protein composed of a plurality of subunits.

### EXAMPLES

### Example 1

### Inoculation (Infiltration) of a Vector Carrying a Gene of Interest (GOP) into a Transgenic Plant Expressing CMV RNA1 (CR1 Tg Plant), and Confirmation of GOP Expression

### (1-1) Preparation of a CR1-Transgenic Plant (CR1 Tg Plant)

(1-1a) Preparation of a CR1-Containing Plasmid and Regeneration of a Plant with the Plasmid

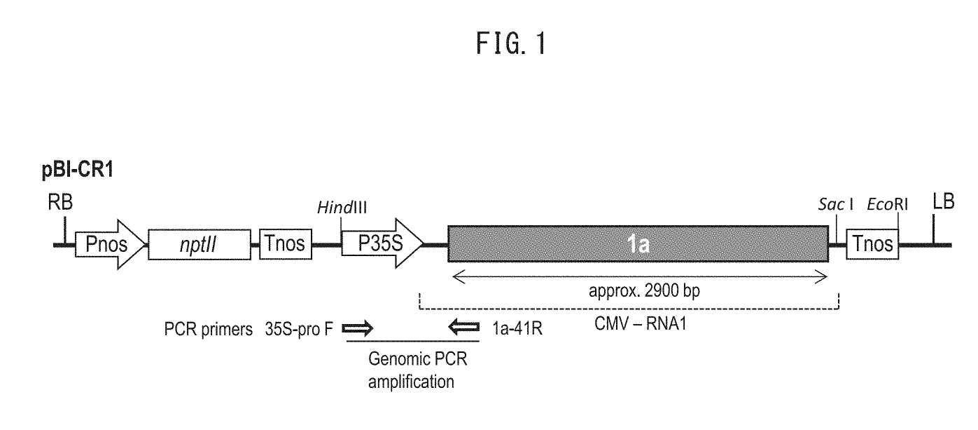

A plasmid pBI-CR1, in which a full-length cDNA of CMV segment genome 1 was inserted into a binary vector pBI121 for plant expression, was constructed as follows. The CMV RNA1 (virus genome) was chosen from the segment genomes of CMV-Y strain and used as a template for amplifying cDNA. The resultant cDNA was then sandwiched between a CMV35S promoter (P35S) and a nopaline synthase terminator (Tnos), and the resultant construct was inserted into pBI121 to prepare plasmid pBI-CR1. In all of the experiments explained below, ligation was carried out using an In-fusion kit (Takara-Bio). The construction of pBI-CR1 is schematically illustrated by FIG. 1.

The resultant plasmid pBI-CR1 was then introduced into Agrobacterium (Agrobacterium tumefacients LBA4404) by a freeze thaw method (Holsters et al., Mol. Gen. Genet., (1978), 163 (2):181-7). The resultant transformed Agrobacterium cells were applied onto LB culture medium (solid) containing 100 mg/L rifampicin, 300 mg/L streptomycin, and 50 mg/L kanamycin, and cultured at 28° C. for two nights to thereby yield colonies of the bacterial cells. The resultant colonies were then inoculated into LB culture medium (liquid) containing 100 mg/L rifampicin, 300 mg/L streptomycin, and 50 mg/L kanamycin, and cultured at 28° C. for two nights to thereby yield Agrobacterium suspension containing pBI-CR1.

The resultant Agrobacterium suspension containing pBI-CR1 was used for gene introduction into Nicotiana benthamiana tobacco plant by a leaf disc method (Horsch et al., Science, (1984), 223: 496-498). Specifically, a leaf disc with a diameter of 1 cm was cut out from a plant leaf, dipped into Agrobacterium suspension containing pBI-CR1 and co-cultured on MS (Murashige-Skoog) agar culture medium for two days. On the third day, the co-cultured leaf disc was washed to remove the sticking bacterial suspension, and then cultured on MS culture medium for redifferentiation containing 50 mg/L kanamycin and 500 mg/L carbenicillin (at 23° C., with a cycle of a 16-hour light period and an 8-hour dark period), and subcultured every two weeks. The shoot obtained via redifferentiation was then cultured on MS culture medium for rooting containing 50 mg/L kanamycin and 500 mg/L carbenicillin to thereby induce rooting, and the cultured individual was conditioned in a glasshouse for Gene modification plants (transgenic plants) and then subjected to soil culture to yield next-generation seeds (T1 individual).

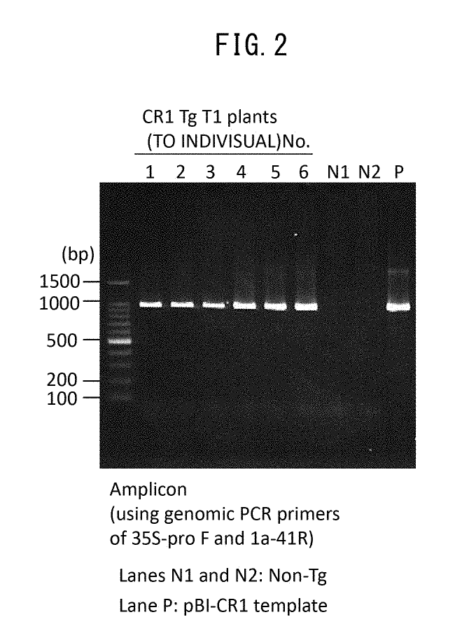

The individuals (T0 and T1 individuals) were cultured for redifferentiation, and leaves were collected from the redifferentiated individuals and subjected to extraction of genome DNA, which was then used as templates for carrying out genomic PCR to confirm gene introduction. CMV RNA1 was detected using a primer pair consisting of 35S-pro F primer (SEQ ID NO: 1: 5′-AGATTAGCCTTTTCAATTTCAGAAAG-3′) and 1a-41 R primer (SEQ ID NO: 2: 5′-CCGTGGGAGGCTACCAATTCATTG-3′). Including all of the experiments explained below, PCR was carried out (unless otherwise specified) using a La Taq (Takara Co.) for amplification. The positions in the plasmid pBI-CR1 corresponding to the 35S-pro F and 1a-41R primers are indicated in FIG. 1. The resultant PCR product was separated on agarose gel, and the separated bands were detected with ethidium bromide. A photograph showing the results of genomic PCR analysis on a T1 individual is indicated in FIG. 2.

The resultant next-generation seeds (T1 individual) were sown on sterile MS (Murashige-Skoog) agar culture medium containing 125 mg/L kanamycin, and individuals exhibiting kanamycin resistance were screened. The resultant individuals were then transplanted to Jiffy7 soil for seeding (Sakata Seed, Co.), and grown for one month in a glasshouse to yield CR1-transgenic plants expressing CMV RNA1.

### (1-1b) Screening for a Transgenic Plant Expressing CR1 (CR1 Tg Plant) and Functionality Evaluation Thereof

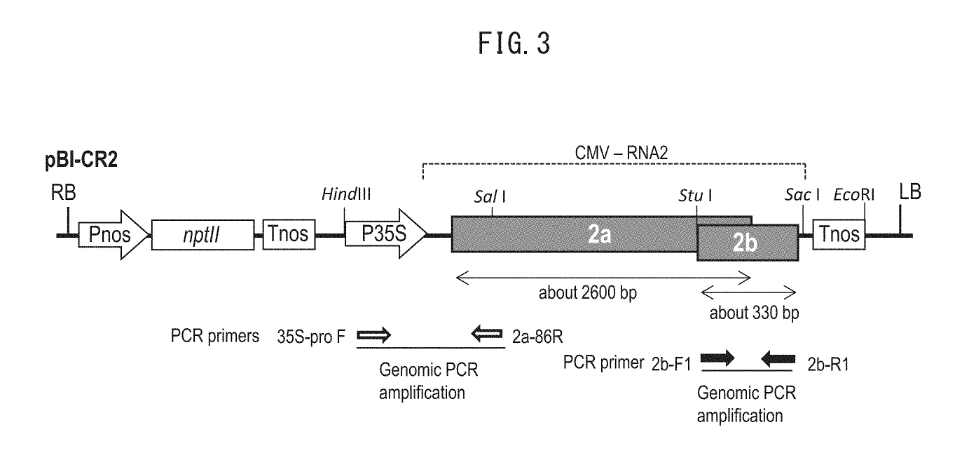

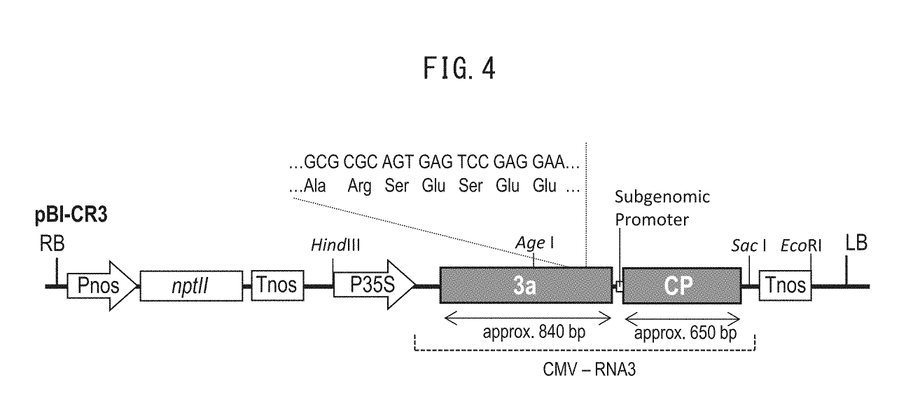

Plasmids pBI-CR2 and pBI-CR3, in which a full-length cDNA of CMV segment genome 2 or 3, respectively, was inserted into a plant expression binary vector pBI121, were constructed as follows. The CMV RNA2 and RNA3 (virus genomes) were chosen from the segment genomes of CMV-Y strain and used as templates for amplifying cDNAs. Each of the resultant cDNAs was then ligated between a CMV35S promoter (P35S) [symbol] nopaline synthase terminator (Tnos), and the resultant construct was inserted into pBI121 to prepare plasmids pBI-CR2 and pBI-CR3. The construction of pBI-CR2 is schematically illustrated in FIG. 3, and the construction of pBI-CR3 is schematically illustrated in FIG. 4.

The resultant pBI-CR2 and pBI-CR3 were each introduced into Agrobacterium (Agrobacterium tumefacients LBA4404) by the method described in section (1-1a) above, to thereby yield Agrobacterium suspension containing pBI-CR2 and Agrobacterium suspension containing pBI-CR3.

Each of the resultant bacterial suspension containing pBI-CR2 and bacterial suspension containing pBI-CR3 was further cultured until the absorbance OD₆₀₀ reaches 0.6, followed by centrifugation (20° C., 4800 rpm, 15 min) to thereby yield a pellet of bacterial cells. The resultant pellets of bacterial cells harboring either pBI-CR2 or pBI-CR3 were suspended in MES buffer (final concentration: 10 mM MES, 10 mM MgCl₂, and 150 μM acetosyringone; pH5.7) such that the resultant OD₆₀₀ value was 0.3 to 0.6, and proliferated until OD₆₀₀ reaches a value between 0.8 and 1.2. The resultant bacterial suspensions were mixed in equal amounts such that the total concentration of the bacterial cells in the final suspension corresponded to an OD600 value of from 0.8 to 1.2 (i.e., the concentrations of the bacterial cells harboring pBI-CR2 and the pBI-CR3—each corresponded to an OD600 value of from 0.4 to 0.8).

The resultant suspension of the bacterial cells harboring pBI-CR2 and the pBI-CR3 in equal amounts was inoculated into the CR1-transgenic plants expressing CMV RNA1 from section (1-1a) above in the following manner. CR1-transgenic plants cultured for 6 to 9 weeks were each placed in a vacuum desiccator, which was depressurized to 0.9 MPa and then instantly returned to ordinary pressure, such that the bacterial cell suspension was forcibly injected (agroinfiltrated) into the plant. After the agroinfiltration, the plants were grown in a plant growth incubator with an artificial weather controller or with a light at 23° C. with a cycle of a 16-hour light period and an 8-hour dark period.

As a result, the plants obviously exhibited a symptom of infection 14 days after the inoculation. Plant lines which exhibited an especially serious symptom of infection showed that functional CMV RNA1 was expressed in the plants and complemented in trans with CMV RNA2 and CMV RNA3 provided by pBI-CR2 and pBI-CR3, respectively, which were inoculated via agroinfiltration, thereby causing proliferation of CMV. Accordingly, these transgenic plant lines were selected and subjected to the inoculation test explained below.

(1-2) Inoculation of a Vector Carrying GFP into the CR1-Transgenic Plant (CR1 Tg Plant) and Confirmation of GFP Expression

A green fluorescent protein (GFP) gene derived from Aequorea victoria (gene length: about 750 bp), which is also used as a marker gene, was used as a gene of interest (GOI). Establishment of a vector carrying GFP, inoculation of the vector into the CR1-transgenic plant (CR1 Tg plant), and confirmation of GFP expression were carried out in the following manner.

### (1-2a) Establishment of a Vector Carrying GFP

Plasmid pBI-CR3, which was established in section (1-1b) above, was used for establishing vectors pBI121-CR3Δ33delGOI and pBI121-CR3Δ33stopGOI, by inserting the gene of interest (GOI) into the region coding for the coat protein (CP) thereof, in the following manner.

Plasmid pCY3 has a backbone based on pUC118, which contains a part of a CMV vector. A 99 bp gene region of the 3a gene of CMV-Y strain at the C-terminal, encodes 33 amino acid residues, was deleted from plasmid pCY3 to yield plasmid pCY3-CR3Δ33delCP. Specifically, pCY3 was used as a template for PCR amplification using a primer pair consisting of MP-Δ33(d) F primer (SEQ ID NO: 3: 5′-TCAGAATGCGCGCAGTTAGCACTTTGGTGCGT-3′) and MP-Δ33(d) R primer (SEQ ID NO: 4: 5′-ACGCACCAAAGTGCTAACTGCGCGCATTCTGA-3′). In this step PCR was carried out using a QuickChange Lighting site-direct Mutagenesis kit (Agilent, Stratagene). The resultant PCR product was digested with restriction enzyme DpnI, and then transformed into competent cells (XL10-Gold) to thereby yield pCY3-CR3Δ33delCP.

On the other hand, plasmid pCY3-CR3Δ33stopCP was prepared such as to express full-length RNA of the 3a gene of the CMV-Y strain, but to be translated into a defect 3a protein lacking 33 amino acid residues at the C-terminal. Specifically, pCY3 was used as a template for PCR amplification using a primer pair consisting of MP-Δ33(s) F primer (SEQ ID NO: 5: 5′-GATTAATCAGAATGCGCGCAGTTAGTCCGAGGAA-3′) and MP-Δ33(s) R primer (SEQ ID NO: 6: 5′-TTCCTCGGACTAACTGCGCGCATTCTGATTAATC-3′). In this step PCR was carried out using a QuickChange Lighting site-direct Mutagenesis kit (Agilent, Stratagene). The resultant PCR product was digested with restriction enzyme DpnI, and then transformed into competent cells (XL10-Gold) to thereby yield pCY3-CR3Δ33stopCP.

Next, the 3b gene of each of the resultant pCY3-CR3Δ33delCP and pCY3-CR3Δ33stopCP vectors was replaced with the GFP gene, which was the gene of interest (GOI), to thereby yield the pCY3-CR3Δ33delGFP and pCY3-CR3Δ33stopGFP vectors.

Specifically, the GFP gene was used as a template for PCR amplification using a primer pair consisting of

CP-GFP F primer
(SEQ ID NO: 7:
5′-GAATTGAGTCGAGTCATGAGTAAAGGAGAAGAAC-3′)
and
CP-GFP R primer
(SEQ ID NO: 8:
5′-TCTGGGAACACGGAATTATTTGTATAGTTCATCC-3′),

to thereby produce cDNA of the GFP gene with a length of about 750 bp. On the other hand, each of the pCY3-CR3Δ33delCP and pCY3-CR3Δ33stopCP vectors produced above were used as a template for PCR amplification using a primer pair consisting of in-fCPVec F primer (SEQ ID NO: 9: 5′-TTCCGTGTTCCCAGAATC-3′) and in-fCPVec R primer (SEQ ID NO: 10: 5′-GACTCGACTCAATTCTAC-3′). The resultant PCR amplification products from these vectors were each ligated with the cDNA of the GFP gene using In Fusion Dry-down PCR cloning Kit (Clontec) and then transformed into competent cells (JM109), to thereby yield clones of pCY3-CR3Δ33delGFP and pCY3-CR3Δ33stopGFP.

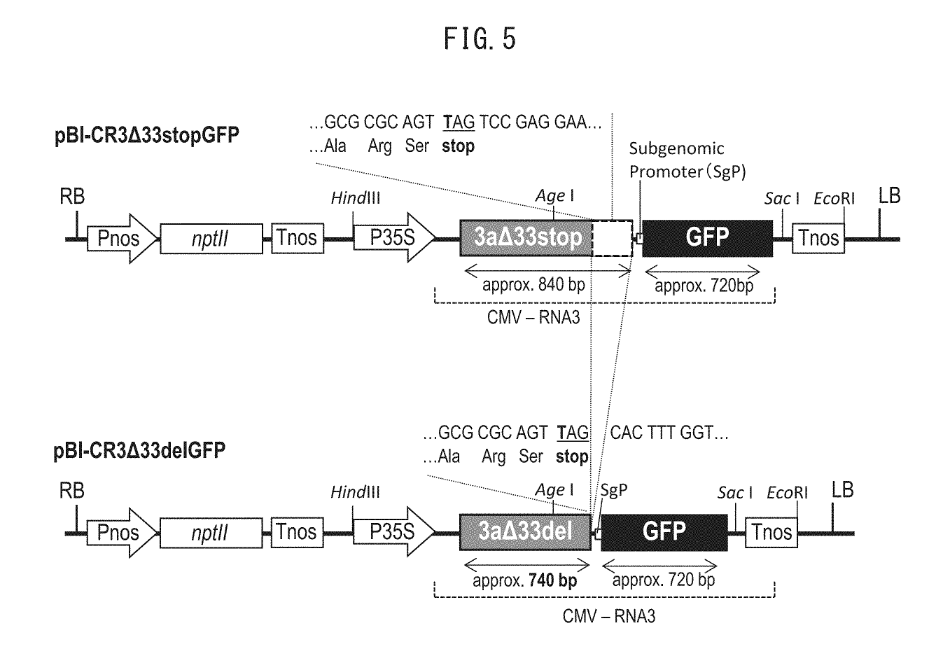

The resultant clones of pCY3-CR3Δ33delGFP and pCY3-CR3Δ33stopGFP were digested with a mixture of restriction enzymes AgeI and SacI to prepare AgeI-Δ33delGFP-SacI fragments and AgeI-Δ33stopGFP-SacI fragments, respectively. The pBI-CR3 plasmid from section (1-1b) above was also linearized with a mixture of restriction enzymes AgeI and SacI. The linearized pBI-CR3 was ligated with each of the AgeI-Δ33delGFP-SacI fragments and the AgeI-Δ33stopGFP-SacI fragments to thereby establish pBI-CR3Δ33delGFP and pBI-CR3Δ33stopGFP. The sequences of the resultant vectors and inserts were confirmed as including no errors, based on analysis of restriction enzyme sites and sequencing with an ABI PRISM Big Dye Terminator (Applied Biosystems, USA). The constructions of the resultant pBI-CR3Δ33delGFP and pBI-CR3Δ33stopGFP binary vectors are schematically illustrated in FIG. 5.

The resultant either pBI-CR3Δ33delGFP or pBI-CR3Δ33stopGFP binary vectors were each introduced into Agrobacterium (LBA4404) by the method described in section (1-1b) above to thereby prepare suspension of bacterial cells.

(1-2b) Infiltration of the Vector Carrying GFP into the Plant and Cultivation of the Infiltrated Plant

The bacterial suspension of Agrobacterium (LBA4404) containing the binary vector pBI-CR3Δ33delGFP or pBI-CR3Δ33stopGFP from section (1-2a) above and the bacterial suspension of Agrobacterium (LBA4404) containing the plasmid pBI-CR2 from section (1-1b) above were each cultured until the absorbance OD₆₀₀ value reached 0.6, followed by centrifugation (20° C., 4800 rpm, 15 min) to thereby prepare a pellet of bacterial cells. The resultant pellets of bacterial cells containing either pBI-CR3Δ33delGFP or pBI-CR3Δ33stopGFP and bacterial cells containing pBI-CR2 were each suspended in MES buffer (final concentration: 10 mM MES, 10 mM MgCl₂, 150 μM acetosyringone, pH5.7) such that the OD₆₀₀ value was between 0.3 to 0.6, and then proliferated until the OD₆₀₀ reached a value between 0.8 and 1.2. These bacterial suspensions were mixed in equal amounts such that the total concentration of bacterial cells in the final suspension corresponded to an OD₆₀₀ value of from 0.8 to 1.2 (i.e., the concentrations of bacterial cells containing the pBI-CR3Δ33stopGFP- and the bacterial cells harboring pBI-CR2 each corresponded to an OD₆₀₀ value of from 0.4 to 0.8).

The resultant suspension of bacterial cells harboring pBI-CR2 and either the pBI-CR3Δ33delGFP- or pBI-CR3Δ33stopGFP in equal amounts was inoculated into the CR1-transgenic plants expressing CMV RNA1 from section (1-1a) above in the following manner. CR1-transgenic plants cultured for 6 to 9 weeks were each placed in a vacuum desiccator, which was depressurized to 0.9 MPa and then instantly returned to ordinary pressure, such that the bacterial cell suspension was forcibly injected (agroinfiltrated) into the plant. After the agroinfiltration, the plants were each grown in a plant growth incubator with an artificial weather controller or with a light at 23° C. with a cycle of a 16-hour light period and an 8-hour dark period.

(1-2c) Confirmation of GFP Expression in the CR1-Transgenic Plant Infiltrated with the Vector Carrying GFP

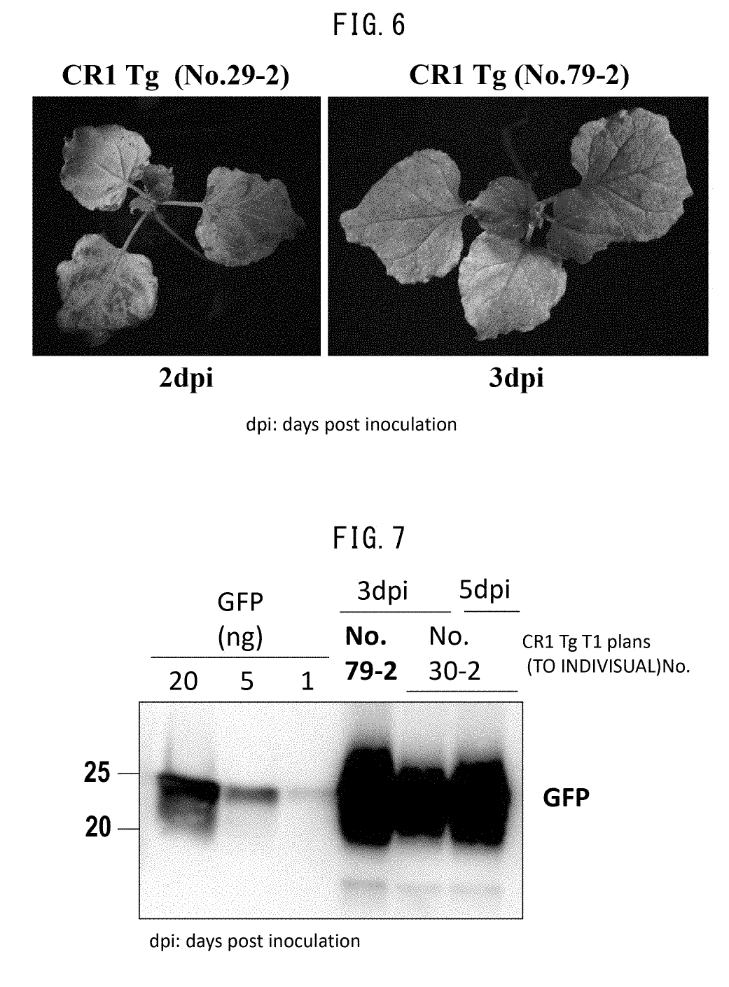

The CR1-transgenic plants inoculated bacterial cells harboring pBI-CR2 and the either pBI-CR3Δ33delGFP- or pBI-CR3Δ33stopGFP via agroinfiltration in section (1-2b) above were subjected to collection of leaves for detection of the target protein. Detection of GFP, which was the gene of interest, was carried out by growing the plants for 3 to 7 days, irradiating the grown plants with blue light having wavelengths that can visualize GFP, and observing the fluorescence from GFP. As a result, GFP fluorescence was observed from the CR1-transgenic plants. A photograph indicating GFP fluorescence from a CR1-transgenic plant obtained using pBI-CR3Δ33delGFP as a vector is indicated in FIG. 6. The results clearly show that functional CMV RNA1 was expressed in the plant and complemented in trans with CMV RNA2 and CMV RNA3a provided by pBI-CR2 and pBI-CR3Δ33delGFP, respectively, which were inoculated via agroinfiltration, thereby causing proliferation of CMV and production of GFP infiltrated by pBI-CR3Δ33delGFP.

In addition, leaves were collected from these plants, crushed with liquid nitrogen, combined with PBS buffer containing 0.1% TritonX100 and a protease inhibitor, and subjected to grinding. The liquid after grinding was centrifuged (4° C., 12000 rpm, 10 min), and the supernatant was subjected to SDS-PAGE. The separated products were transferred to a PVDF membrane, and subjected to western blot analysis using an anti-GFP antibody. The results of western blot analysis result obtained by using pBI-CR3Δ33delGUS as a vector are shown in FIG. 7. The detected bands were subjected to densitometric analysis using Gel-doc from BIO-RAD. As a result, it was confirmed that 750 mg of GFP protein was expressed and accumulated from 1 kg of leaves of the plant three days after the inoculation.

(1-3) Inoculation of a Vector Carrying GUS (Long-Chain Gene) into the CR1-Transgenic Plant (CR1 Tg Plant) and Confirmation of GUS Expression

β-glucuronidase (GUS) gene (gene length: about 1810 bp), which is a long-chain gene also used as a marker gene, was used as a gene of interest (GOI). Establishment of a GUS-containing vector, inoculation of the vector into the CR1-transgenic plant (CR1 Tg plant), and confirmation of GUS expression were carried out in the following manner.

As constructs for expressing the GUS gene, pCY3-CR3Δ33delGUS and pCY3-CR3Δ33stopGUS vectors were established by replacing the 3b gene of each of the pCY3-CR3Δ33delCP and pCY3-CR3Δ33stopCP vectors from section (1-2a) above with the GUS gene. Specifically, the GUS gene was subjected to PCR amplification using a primer pair consisting of

FW CP-GUS primer
(SEQ ID NO: 11:
5′-GAATTGAGTCGAGTCATGTTACGTCCTGTAGAAAC-3′)
and
RV CP-GUS primer
(SEQ ID NO: 12:
5′-TCTGGGAACACGGAAATTGTTTGCCTCCCTGCTGC-3′),

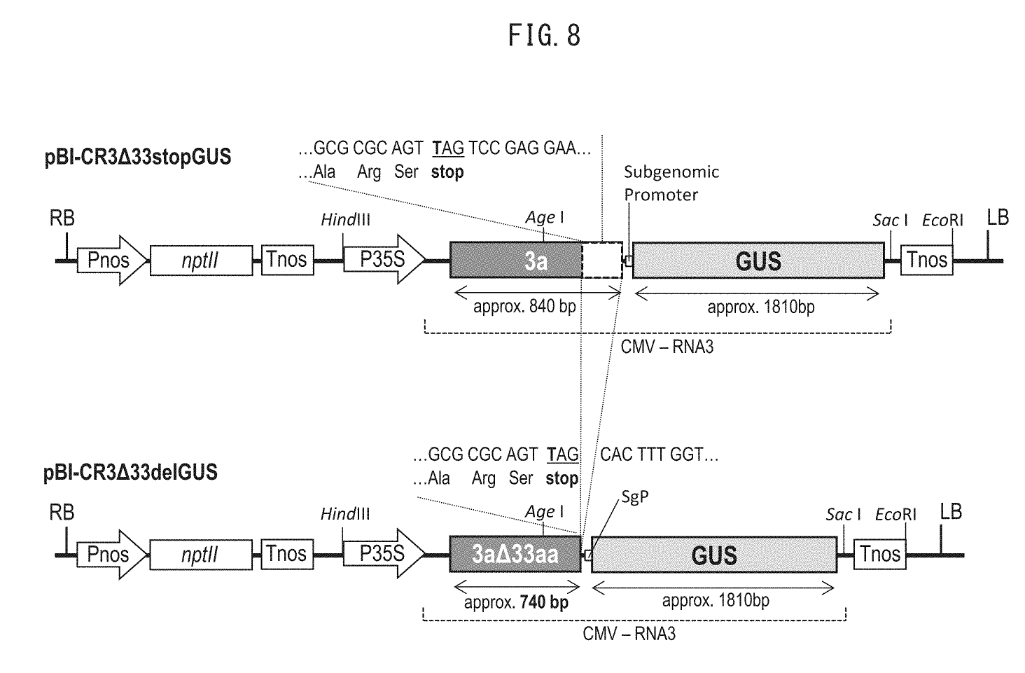

to thereby yield cDNA of the GUS gene with a length of about 1810bp. Except that the resultant cDNA of the GUS gene was used instead of the cDNA of the GFP gene in section (1-2a) above, the same procedure as that described in section (1-2a) above was carried out to produce plasmids pCY3-CR3Δ33delGUS and pCY3-CR3Δ33stopGUS, in which the 3b gene of each of the pCY3-CR3Δ33delCP and pCY3-CR3Δ33stopCP vectors, respectively, was replaced with the GUS gene. These plasmids were linearized by the method described in section (1-2a) above, and ligated with pBI-CR3 which had been linearized in the same manner, using an In-Fusion Dry-down PCR cloning Kit (Clontec), to thereby prepare pBI-CR3Δ33delGUS and pBI-CR3Δ33stopGUS. The sequences of the resultant vectors and inserts were confirmed as including no errors, in the same manner as described in section (1-2a) above. The constructions of the resultant pBI-CR3Δ33delGUS and pBI-CR3Δ33stopGUS vectors are schematically illustrated in FIG. 8. The resultant pBI-CR3Δ33delGUS and pBI-CR3Δ33stopGUS binary vectors were each introduced into Agrobacterium (LBA4404) by the method described in section (1-1b) above to thereby prepare bacterial suspension.

The resultant pBI-CR3Δ33delGUS and pBI-CR3Δ33stopGUS were each introduced into Agrobacterium (LBA4404) by the method described in section (1-1b) above to thereby prepare bacterial suspension.

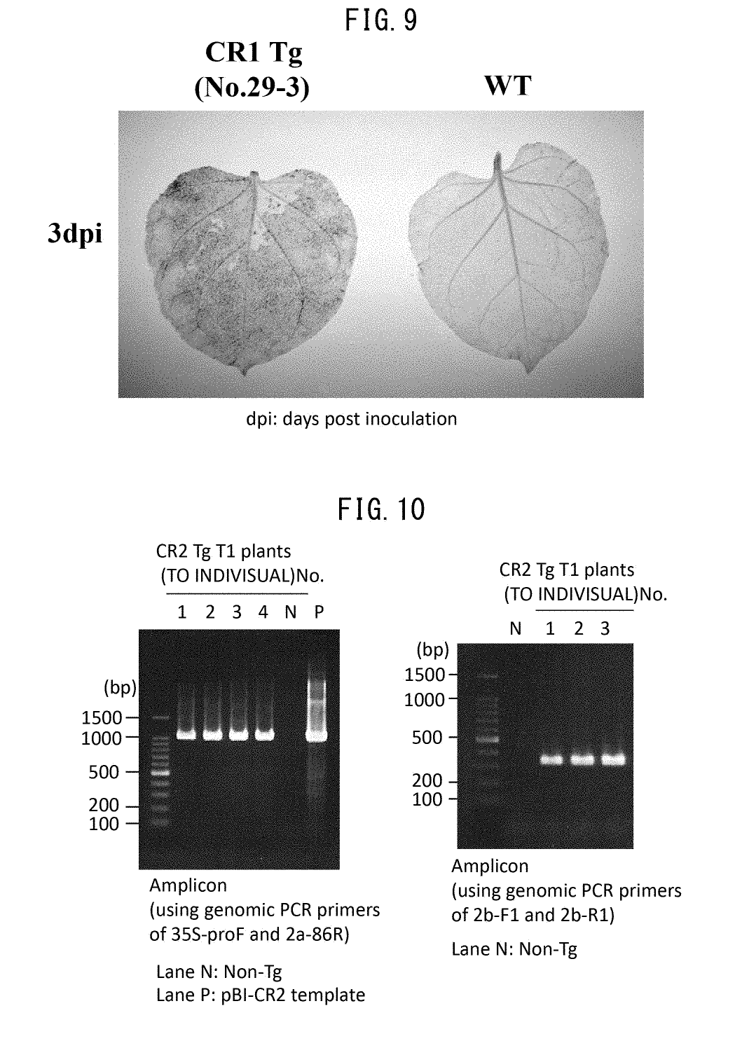

Next, except that bacterial suspension of Agrobacterium (LBA4404) containing the binary vector pBI-CR3Δ33delGUS or pBI-CR3Δ33stopGUS was used, the procedure described in section (1-2b) above was carried out to thereby forcibly inject (agroinfiltrate) the bacterial cells suspension into the CR1-transgenic plants expressing CMV RNA1 from section (1-1b) above. After the agroinfiltration, leaves were collected from the CR1-transgenic plants and subjected to detection of the target protein (GUS). Detection of GUS was carried out by dissolving 10 mg of 5-bromo-4-chloro-3-indolyl-beta delta-glucuronic acid (X-Gluc) powder, which is a chromogenic substrate of GUS, into 100 μL of dimethyl formamide, which was then diluted ten times with 50 mM phosphoric acid sodium buffer (pH7.0), to which the leaves were added and incubated at 37° C. After the color of GUS was confirmed, the solvent was exchanged with 70% ethanol to decolorize chloroplasts. As a result, GUS expression was confirmed in the CR1-transgenic plants. A photograph indicating the color obtained by using pBI-CR3Δ33delGUS as the GUS vector is shown in FIG. 9. The results clearly show that functional CMV RNA1 was expressed in the plant and complemented in trans with CMV RNA2 and CMV RNA3a provided by pBI-CR2 and pBI-CR3Δ33delGUS, respectively, which were inoculated via agroinfiltration, thereby causing proliferation of CMV and production of GUS introduced by pBI-CR3Δ33delGUS.

### Example 2

### Inoculation of a Vector Carrying a Gene of Interest (GOP) into a Transgenic Plant Expressing CMV RNA2 (CR2 Tg Plant) and Confirmation of GOP Expression

### (2-1) Preparation of a CR2-Transgenic Plant (CR2 Tg Plant)

Except that the plasmid pBI-CR2 produced in section (1-1b) above was used instead of plasmid pBI-CR1, the procedure described in section (1-1a) above was carried out to yield next-generation seeds (T1 individual) of a CR2-transgenic plant (CR2 Tg plant).

The individuals (T0 and T1 individuals) were cultured for redifferentiation, and leaves were collected from the redifferentiated individuals and subjected to extraction of genome DNA, which was then used as templates for carrying out genomic PCR to confirm gene introduction. A region at the 5′ side of CMV RNA2 was detected using a primer pair consisting of 35S-pro F primer (SEQ ID NO: 1: 5′-AGATTAGCCTTTTCAATTTCAGAAAG-3′) and 2a-86 R primer (SEQ ID NO: 13: 5′-AAACGTTCCACATCCTCGGGAGT-3′). A region at the 3′ side of CMV RNA2 was detected using a primer pair consisting of 2b-F1primer (SEQ ID NO: 14: 5′-ATGGAATTGAACGTAGGTGCAATG-3′) [symbol] 2b-R1primer (SEQ ID NO: 15: 5′-TCAGAAAGCACCTTCCGCC-3′). The positions in the plasmid pBI-CR2 corresponding to the 35S-pro F and 2a-86R primers and the 2b-F1 and 2b-R1 primers are indicated in FIG. 3. The remaining steps were carried out in the same manner as in section (1-1b) above. A photograph showing the results of genomic PCR analysis on a T1 individual is indicated in FIG. 10.

The resultant next-generation seeds (T1 individual) were screened for individuals having kanamycin resistance in the same manner as described in section (1-1b) above, and the selected individuals were grown for one month in a glasshouse to thereby yield CR2-transgenic plants expressing CMV RNA2.

On the other hand, except that pBI-CR1 shown in FIG. 1 was used instead of pBI-CR2 shown in FIG. 3, the procedure described in section (1-1b) above was carried out to thereby yield bacterial suspension of Agrobacterium containing pBI-CR1 and bacterial suspension containing pBI-CR3.

The resultant bacterial suspensions were forcibly injected (agroinfiltrated) into the CR2-transgenic plants in the same manner as described in section (1-1b) above. Seven days after the infiltration, infiltrated transgenic plant lines which exhibited an especially serious symptom of infection showed that functional CMV RNA2 was expressed in the plants and complemented in trans with CMV RNA1 and CMV RNA3 provided by pBI-CR1 and pBI-CR3, respectively, which were infiltrated via agroinfiltration, thereby causing proliferation of CMV. Accordingly, these infiltrated transgenic plant lines were selected and subjected to the inoculation test explained below.

(2-2) Inoculation of a Vector Carrying GFP into the CR2-Transgenic Plant (CR2 Tg Plant) and Confirmation of GFP Expression

Establishment of a vector containing GFP as the gene of interest (GOI) and production of bacterial suspension of Agrobacterium cells into which the vector was introduced were carried out in the same manner as described in section (1-2a) above. Next, except that the CR2-transgenic plants expressing CMV RNA2 (CR2 Tg plants) produced in section (2-1) above were used instead of the CRltransgnic plants expressing CMV RNA1 (CR1 Tg plants), and that the bacterial suspension of Agrobacterium containing plasmid pBI-CR1 produced in section (1-1b) above was used instead of the bacterial suspension of

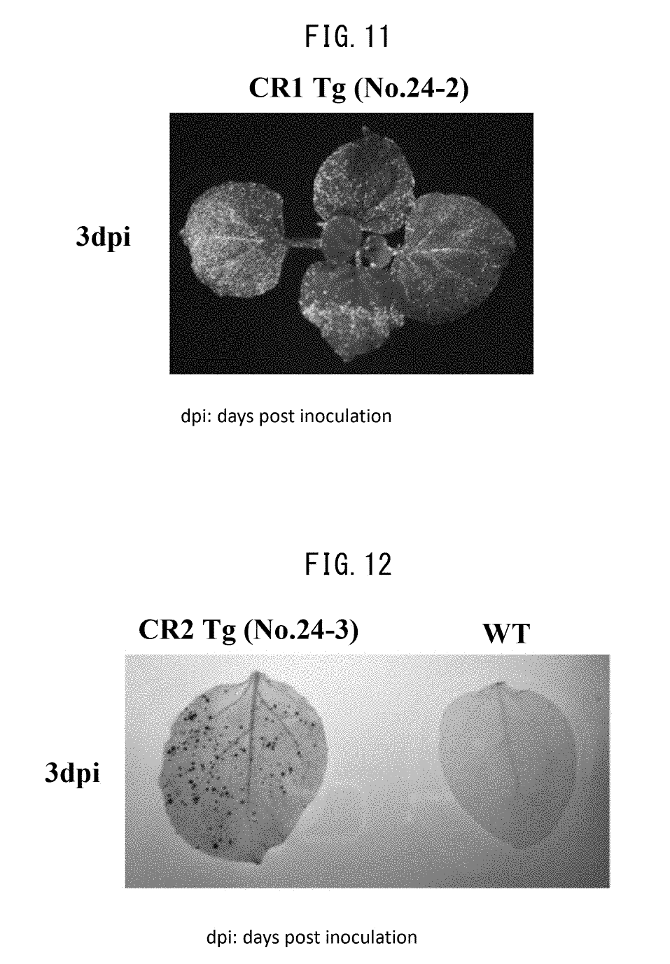

Agrobacterium containing of plasmid pBI-CR2, infiltration of the bacterial suspension into the CR2-transgenic plants (CR2 Tg plants) and confirmation of GFP expression were carried out in the same manner as described in section (1-2a) above. As a result, GFP fluorescence was observed in the CR2-transgenic plants (CR2 Tg plants). A photograph indicating the GFP fluorescence obtained by using pBI-CR3Δ33delGUS as a binary vector is shown in FIG. 11.

(2-3) Inoculation of a Vector Carrying GUS (Long-Chain Gene) into the CR2-Transgenic Plants (CR2 Tg Plants) and Confirmation of GFP Expression

Establishment of a vector containing GUS as the gene of interest (GOI) and production of bacterial suspension of Agrobacterium into which the vector was introduced were carried out in the same manner as described in section (1-3) above. Next, except that the CR2-transgenic plants expressing CMV RNA2 (CR2 Tg plants) produced in section (2-1) above instead of the CR1-transgenic plants expressing CMV RNA1 (CR1 Tg plants), and that the bacterial suspension of Agrobacterium containing plasmid pBI-CR1 produced in section (1-1b) above was used instead of the bacterial suspension of Agrobacterium containing plasmid pBI-CR2, infiltration of the bacterial suspension into the CR2-transgenic plants (CR2 Tg plants) and confirmation of GUS expression were carried out in the same manner as described in section (1-3) above. As a result, GUS expression was observed in the CR2-transgenic plants (CR2 Tg plants). A photograph indicating the GUS expression obtained by using pBI-CR3Δ33delGUS as a binary vector is shown in FIG. 12.

### Example 3

### Infiltration of a Vector Carrying a Gene of Interest (GOP) into Transgenic Plants Expressing both CMV RNA1 and RNA2 (CR1+CR2 Tg Plants) and Confirmation of GOP Expression

### (3-1) Preparation of a CR1+CR2-Transgenic Plant (CR1+CR2 Tg Plant)

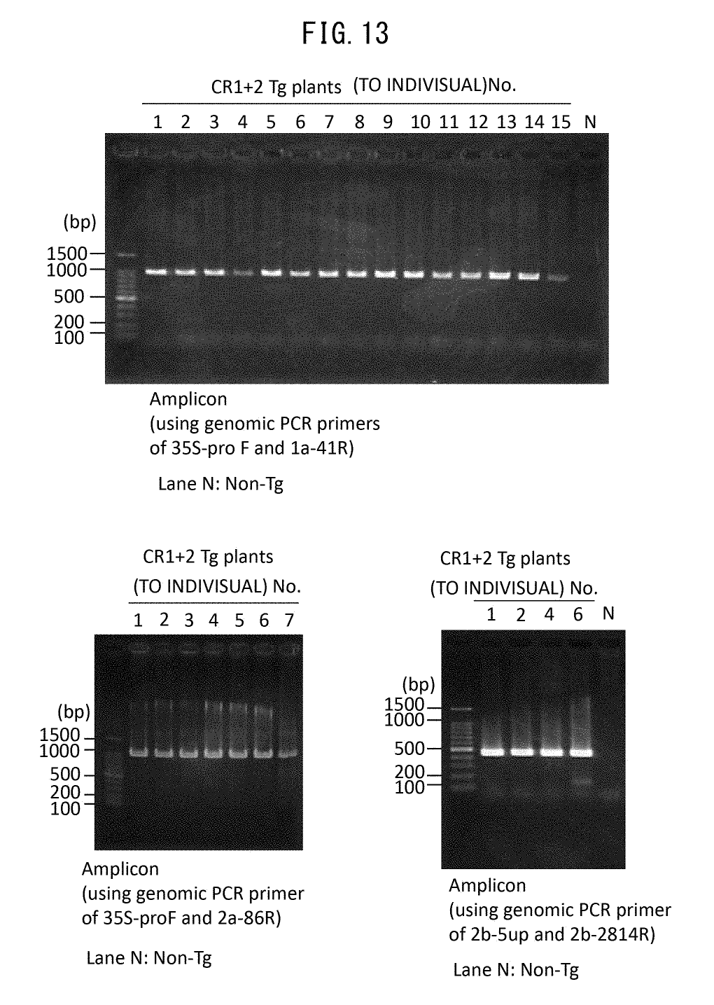

A CR1-transgenic plant produced in section (1-1) above and a CR1-transgenic plant produced in section (2-1) above were crossbred, and the resultant individual plants were grown and cross-pollinated at the time of bloom to thereby produce transgenic plants expressing both CMV RNA1 and RNA2 (CR1+2 Tg). Next, the resultant seeds were sown on sterile MS culture medium containing 125 mg/L kanamycin, and individuals having kanamycin resistance were selected. Leaves were collected from the selected individuals and subjected to extraction of genome DNA, which was used as templates in genomic PCR for confirming gene introduction. Detection of CMV RNA1 was carried out using the 35S-pro F primer (SEQ ID NO: 1) and the 1a-41 R primer (SEQ ID NO: 2) described in section (1-1a) above. Detection of a region at the 5′ side of CMV RNA2 was carried out using the 35S-pro F primer (SEQ ID NO: 1) and the 2a-86 R primer (SEQ ID NO: 13) described in section (2-1) above. Detection of a region at the 3′ side of CMV RNA2 was carried out using 2b-5upprimer (SEQ ID NO: 16: 5′-GTACAGAGTTCAGGGTTGAGCG-3′) and 2b-2814 R primer (SEQ ID NO: 17: 5′-AGCAATACTGCCAACTCAGCTCC-3′). A photograph showing the results of genomic PCR analysis on these individual is shown in FIG. 13. These individuals were screened for transgenic tobacco plants expressing both CMV RNA1 and CMV RNA 2.

On the other hand, except that only the pBI-CR3 shown in FIG. 4 was used while the pBI-CR2 shown in FIG. 3 was not used, the procedure described in section (1-1b) above was carried out to yield bacterial suspension of Agrobacterium containing pBI-CR3.

The resultant bacterial suspension was forcibly injected (agroinfiltrated) into the CR1+CR2-transgenic plants in the same manner as described in section (1-1b) above. Seven days after the infiltration, infiltrated transgenic plant lines which exhibited a large amount of CMV proliferation showed that functional CMV RNA1 and CMV RNA2 were expressed and in the plants and complemented in trans with CMV RNA3 provided by pBI-CR3, which was inoculated via agroinfiltration, thereby causing proliferation of CMV. Accordingly, these transgenic plant lines were selected and subjected to the inoculation test explained below.

(3-2) Inoculation of a Vector Carrying GFP into the CR1+CR2-Transgenic Plant (CR1+CR2 Tg Plant) and Confirmation of GFP Expression

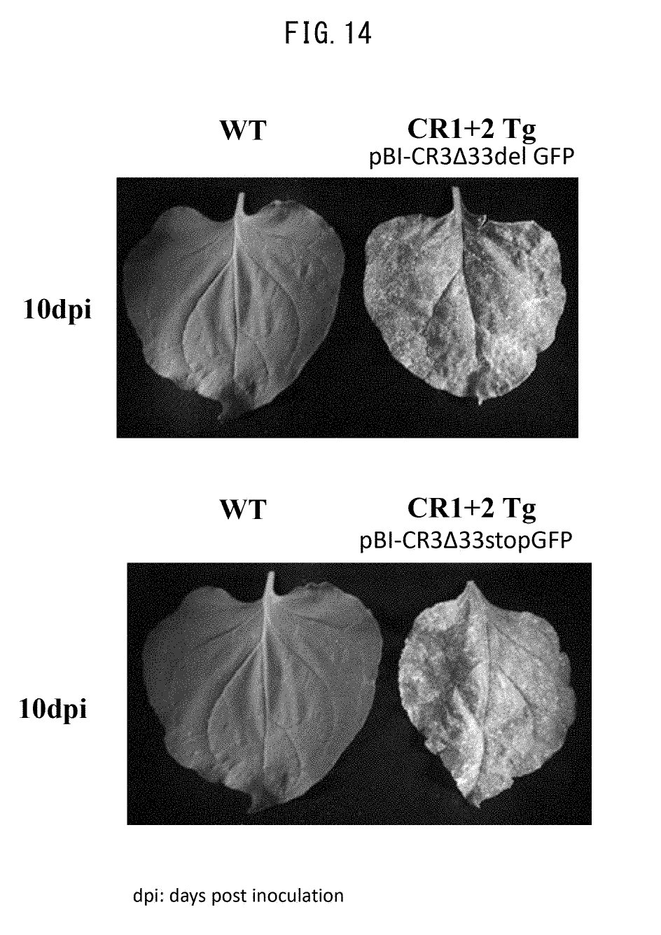

Establishment of a vector containing GFP as the gene of interest (GOI) and production of bacterial suspension of Agrobacterium into which the vector was introduced were carried out in the same manner as described in section (1-2a) above. Next, except that the CR1+CR2-transgenic plants expressing CMV RNA1 and RNA2 (CR1+CR2 Tg plants) produced in section (3-1) above were used instead of the CR1-transgenic plants expressing CMV RNA1 (CR1 Tg plants), and that the bacterial suspension of Agrobacterium containing plasmid pBI-CR2 was not used, inoculation of the bacterial suspension into the CR1+CR2-transgenic plants (CR1+CR2 Tg plants) and confirmation of GFP expression were carried out in the same manner as described in section (1-2a) above. As a result, GFP fluorescence was observed in the CR1+CR2-transgenic plants (CR1+CR2 Tg plants). Photographs indicating the GFP fluorescence obtained by using either pBI-CR3Δ33delGUS or pBI-CR3Δ33stopGUS as binary vectors are shown in FIG. 14.

(3-3) Inoculation of a Vector Carrying GUS (Long-Chain Gene) into the CR1+CR2-Transgenic Plants (CR1+CR2 Tg Plants) and Confirmation of GUS Expression

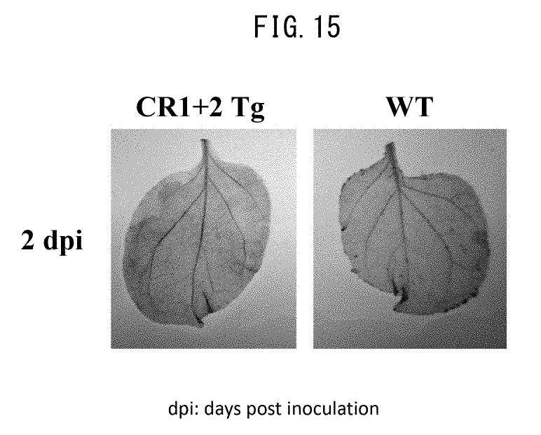

Establishment of a vector containing GUS as the gene of interest (GOI) and production of bacterial suspension of Agrobacterium cells into which the vector was introduced were carried out in the same manner as described in section (1-3) above. Next, except that the CR1+CR2-transgenic plants expressing CMV RNA1 and RNA2 (CR1+CR2 Tg plants) produced in section (3-1) above were used instead of the CR1-transgenic plants expressing CMV RNA1 (CR1 Tg plants), and that the bacterial suspension of Agrobacterium containing plasmid pBI-CR2 was not used, inoculation of the bacterial suspension of into the CR1+CR2-transgenic plants (CR1+CR2 Tg plants) and confirmation of GUS expression were carried out in the same manner as described in section (1-3) above. As a result, GUS expression was observed in the CR1+CR2-transgenic plants (CR1+CR2 Tg plants). A photograph indicating the GUS expression obtained by using pBI-CR3Δ33delGUS as a binary vector is shown in FIG. 15.

1. A method for expressing a foreign gene, comprising the steps of:
(a) producing a plant which functionally expresses an RNA1 genome and/or an RNA2 genome of Cucumber Mosaic Virus (CMV);
(b) introducing a nucleic acid vector into the plant, said nucleic acid vector comprising, arranged in this order:
(i) a right border sequence (RB) derived from the Agrobacterium T-DNA sequence;
(ii) an expression promoter sequence workable in the plant;
(iii) a sequence corresponding to an RNA3 genome of CMV in which a 3b gene, which codes for a 3b protein (coat protein) of CMV has been prevented from being expressed and the foreign gene has been inserted downstream of, and operably linked with, a subgenomic promoter which otherwise promotes the expression of the 3b gene; and
(iv) a left border sequence (LB) derived from the Agrobacterium T-DNA sequence; and
(c) culturing the plant containing the nucleic acid vector such that the RNA1 and/or RNA2 genome(s) of CMV is functionally expressed by the plant while the foreign gene is expressed transiently from the nucleic acid vector along with the CMV RNA3 genome lacking a region corresponding to the 3b gene.

2. The method according to claim 1,
wherein the plant produced in said step (a) functionally expresses the CMV RNA1 genome,
wherein said step (b) includes introducing, together with the nucleic acid vector, a nucleic acid molecule CR2 into the plant, said nucleic acid molecule CR2 comprising, arranged in this order:
a right border sequence (RB) derived from the Agrobacterium T-DNA sequence;
an expression promoter sequence workable in the plant;
a sequence corresponding to the CMV RNA2 genome; and
a left border sequence (LB) derived from the Agrobacterium T-DNA sequence; and
wherein in said step (c), the CMV RNA1 genome is functionally expressed by the plant while the CMV RNA2 genome is expressed transiently from the nucleic acid molecule CR2 in the plant, along with the foreign gene and the CMV RNA3 genome lacking the region corresponding to the 3b gene transiently expressed from the nucleic acid vector.

3. The method according to claim 2, wherein the introduction of the nucleic acid vector and the nucleic acid molecule CR2 into the plant in said step (b) is carried out by infiltrating or injecting a culture solution containing Agrobacterium carrying the nucleic acid vector and Agrobacterium carrying the nucleic acid molecule CR2 into the plant.

4. The method according to claim 1,
wherein the plant produced in said step (a) functionally expresses the CMV RNA2 genome in the plant,
wherein said step (b) includes introducing, together with the nucleic acid vector, a nucleic acid molecule CR1 into the plant, said nucleic acid molecule CR1 comprising, arranged in this order:
a right border sequence (RB) derived from the Agrobacterium T-DNA sequence;
an expression promoter sequence workable in the plant;
a sequence corresponding to the CMV RNA1 genome; and
a left border sequence (LB) derived from the Agrobacterium T-DNA sequence; and
wherein in said step (c), the RNA2 genome of CMV is functionally expressed by the plant while the CMV RNA1 genome is expressed transiently from the nucleic acid molecule CR1 in the plant, along with the foreign gene and the CMV RNA3 genome lacking the region corresponding to the 3b gene transiently expressed from the nucleic acid vector.

5. The method according to claim 4, wherein the introduction of the nucleic acid vector and the nucleic acid molecule CR1 into the plant in said step (b) is carried out by infiltrating or injecting a culture solution containing Agrobacterium carrying the nucleic acid vector and Agrobacterium carrying the nucleic acid molecule CR1 into the plant.

6. The method according to claim 1,
wherein the plant produced in said step (a) functionally expresses the CMV RNA1 genome and the CMV RNA2 genome; and
wherein in said step (c), the RNA1 and RNA2 genomes of CMV are functionally expressed by the plant while the foreign gene and the CMV RNA3 genome lacking the region corresponding to the 3b gene are transiently expressed from the nucleic acid vector.

7. The method according to claim 6, wherein the introduction of the nucleic acid vector into the plant in said step (b) is carried out by infiltrating or injecting a culture solution containing Agrobacterium carrying the nucleic acid vector into the plant.

8. The method according to claim 1, wherein the expression of a region at the C-terminus of a 3a protein that interacts with the expression of the 3b protein is inhibited.

9. The method according to claim 8, wherein the sequence corresponding to the CMV RNA3 genome includes a stop codon inserted in the 3a gene so as to prevent expression of the region at the C-terminus of the 3a protein.

10. The method according to claim 1, wherein the foreign gene has a length of 900 bp or larger.

11. The method according to claim 1, which is in the form of a T-DNA vector.

12. The method according to claim 1, wherein diffusion of the foreign gene to outside the plant is inhibited.

13. An expression system for expressing a foreign gene, comprising:
(A) a plant which functionally expresses at least either an RNA1 genome or an RNA2 genome of Cucumber Mosaic Virus (CMV); and
(B) a nucleic acid vector comprising, arranged in this order:
(i) a right border sequence (RB) derived from the Agrobacterium T-DNA sequence;
(ii) an expression promoter sequence workable in the plant;
(iii) a sequence corresponding to an RNA3 genome of CMV in which a 3b gene, which codes for a 3b protein (coat protein) of CMV, has been prevented from being expressed and the foreign gene has been inserted downstream of, and operably linked with, a subgenomic promoter which otherwise promotes the expression of the 3b gene; and
(iv) a left border sequence (LB) derived from the Agrobacterium T-DNA sequence.

14. The expression system according to claim 13, wherein
said plant of (A) functionally expresses the CMV RNA1 genome, and
said expression system further comprises:
(C) a nucleic acid molecule CR2 comprising, arranged in this order:
a right border sequence (RB) derived from the Agrobacterium T-DNA sequence;
(ii) an expression promoter sequence workable in the plant;
(iii) a sequence corresponding to the CMV RNA2 genome; and
(iv) a left border sequence (LB) derived from the Agrobacterium T-DNA sequence.

15. The expression system according to claim 13, wherein
said plant of (A) functionally expresses the CMV RNA2 genome, and
said expression system further comprises:
(C) a nucleic acid molecule CR1 comprising, arranged in this order:
(i) a right border sequence (RB) derived from the Agrobacterium T-DNA sequence;
(ii) an expression promoter sequence workable in the plant;
(iii) a sequence corresponding to the CMV RNA1 genome; and
(iv) a left border sequence (LB) derived from the Agrobacterium T-DNA sequence.

16. The expression system according to claim 13, wherein said plant of (A) functionally expresses the CMV RNA1 genome and the CMV RNA2 genome.

17. The expression system according to claim 13, wherein the expression of a region at the C-terminus of a 3a protein that interacts with the expression of the 3b protein is inhibited.

18. The expression system according to claim 14, wherein the sequence corresponding to the CMV RNA3 genome includes a stop codon inserted in the 3a gene so as to prevent expression of the region at the C-terminus of the 3a protein.

19. The expression system according to claim 13, wherein the foreign gene has a length of 900 bp or larger.

20. The expression system according to claim 13, which is in the form of a T-DNA vector.


SEQUENCE LISTING (17 sequences)

SEQ ID NO: 1 - DNA, 26 bp, Artificial Sequence
  35S-pro F primer
agattagccttttcaatttcagaaag

SEQ ID NO: 2 - DNA, 24 bp, Artificial Sequence
  1a-41 R primer
ccgtgggaggctaccaattcattg

SEQ ID NO: 3 - DNA, 32 bp, Artificial Sequence
  MP-delta 33(d) F primer
tcagaatgcgcgcagttagcactttggtgcgt

SEQ ID NO: 4 - DNA, 32 bp, Artificial Sequence
  MP-delta 33(d) R primer
acgcaccaaagtgctaactgcgcgcattctga

SEQ ID NO: 5 - DNA, 34 bp, Artificial Sequence
  MP-delta 33(s) F primer
gattaatcagaatgcgcgcagttagtccgaggaa

SEQ ID NO: 6 - DNA, 34 bp, Artificial Sequence
  MP-delta 33(s) R primer
ttcctcggactaactgcgcgcattctgattaatc

SEQ ID NO: 7 - DNA, 34 bp, Artificial Sequence
  FW CP-GFP primer
gaattgagtcgagtcatgagtaaaggagaagaac

SEQ ID NO: 8 - DNA, 34 bp, Artificial Sequence
  RV CP-GUS primer
tctgggaacacggaattatttgtatagttcatcc

SEQ ID NO: 9 - DNA, 18 bp, Artificial Sequence
  in-fCPVec F primer
ttccgtgttcccagaatc

SEQ ID NO: 10 - DNA, 18 bp, Artificial Sequence
  in-fCPVec R primer
gactcgactcaattctac

SEQ ID NO: 11 - DNA, 35 bp, Artificial Sequence
  CP-GUS F primer
gaattgagtcgagtcatgttacgtcctgtagaaac

SEQ ID NO: 12 - DNA, 35 bp, Artificial Sequence
  CP-GUS R primer
tctgggaacacggaaattgtttgcctccctgctgc

SEQ ID NO: 13 - DNA, 23 bp, Artificial Sequence
  2a-86 R primer
aaacgttccacatcctcgggagt

SEQ ID NO: 14 - DNA, 24 bp, Artificial Sequence
  2b-F1 primer
atggaattgaacgtaggtgcaatg

SEQ ID NO: 15 - DNA, 19 bp, Artificial Sequence
  2b-R1 primer
tcagaaagcaccttccgcc

SEQ ID NO: 16 - DNA, 22 bp, Artificial Sequence
  2b-5up primer
gtacagagttcagggttgagcg

SEQ ID NO: 17 - DNA, 23 bp, Artificial Sequence
  2b-2814R primer
agcaatactgccaactcagctcc